(19) United States
(12) Patent Application Publication
(10) Pub. No.: US 2021/0145741 A1
(43) Pub. Date: May 20, 2021

(54) ANTIFIBROTIC COMPOSITION

(71) Applicants: Lamellar Biomedical Limited, Holytown (GB); The University Court of the University of Edinburgh, Edinburgh (GB)

(72) Inventors: Alec McLean, Holytown (GB); Margaret Courtney, Holytown (GB); Lynsey Howard, Holytown (GB); Steven Porteous, Holytown (GB); David Collie, Edinburgh (GB); Gerry McLachlan, Edinburgh (GB)

(21) Appl. No.: 17/048,234

(22) PCT Filed: Apr. 17, 2019
(86) PCT No.: PCT/GB2019/051102

§ 371 Date: Oct. 16, 2020

(30) Foreign Application Priority Data
Apr. 17, 2018 (GB) 1806278.6
Oct. 5, 2018 (EP) 18198987.2

(51) Int. Cl. A61K 9/00; A61K 31/685; A61K 31/575; A61K 31/683; A61K 31/7028; A61K 31/688; A61P 11/00

(52) U.S. Cl. CPC A61K 9/0082; A61K 31/685; A61K 31/575; A61P 11/00; A61K 31/7028; A61K 31/688; A61K 31/683

(57) ABSTRACT

Methods of prophylactically treating fibrotic conditions using a synthetic lamellar body composition are provided, such as conditions of the lung, skin, gastrointestinal system, genitourinary system, heart, peritoneum, kidney, liver, and mucosa. In particular the present invention is concerned with lung injury which may be characterised by increased pulmonary vascular permeability. Suitably the lamellar body composition for use in the prophylactic treatment of fibrotic or pro-fibrotic conditions comprises phosphatidylcholine, cholesterol and optionally at least one phospholipid selected from phosphatidyl serine, phosphatidyl glycerol and phosphatidyl inositol to provide an anionic lamellar body.

### FIELD OF THE INVENTION

The present invention concerns methods of treating aberrant fibrotic conditions, such as conditions of the lung, skin, gastrointestinal system, genitourinary system, heart, peritoneum, kidney, liver, and mucosa. In particular the present invention is concerned with lung injury which may be characterised by increased pulmonary vascular permeability. Suitably, the present invention is related to methods of treating lung parenchyma in order to limit the harmful progression of distal lung injuries or events that are initiated at the level of the alveolar-capillary membrane, and which impact on the functional integrity of the gas exchange surface of the lung to the extent that pulmonary oedema develops and surfactant function is impaired.

### BACKGROUND OF THE INVENTION

Whilst the clinical syndromes of acute lung injury (ALI) and the acute respiratory distress syndrome (ARDS) exemplify such distal lung injury and may be elicited by a range of direct and indirect insults ranging from pneumonia, to sepsis and trauma, another recognised insult that can compromise the alveolar-capillary membrane in this manner is exposure to ionising radiation. Such injuries cause damage that will usually resolve. However, in a proportion of individuals there may instead be further progression to the extent that the alveolar-capillary membrane becomes chronically compromised by the deposition of collagen (alveolar fibrosis) and tissue remodelling. The harmful progression of distal lung injuries or events that are initiated at the level of the alveolar-capillary membrane can lead to progressive alveolar fibrosis and respiratory compromise that can significantly impact on quality of life and lead to respiratory failure. Surfactant is considered to be reduced and/or impaired, in conditions identified as initially impacting on the alveolar-capillary membrane (e.g. ALI/ARDS, Radiation-Induced Lung Injury (RILI)). Surfactant as physiologically produced in the body is understood to provide a monolayer structure on a cell membrane, but the surfactant is not capable of crossing the cell membrane into alveoli and/or the interstitium.

### SUMMARY OF THE INVENTION

ARDS can arise from many causes eg. direct lung injury as a result of the aspiration of stomach contents or indirectly e.g. as a result of severe trauma. In term of ARDS, this precipitates an inflammatory cascade involving the recruitment of potent inflammatory cells such as neutrophils and the secretion of proinflammatory cytokines and chemokines. The initial and function-limiting pathology of the condition occurs in the interstitium and alveolar cells. Whilst physiologically provided surfactant can provide a protective effect to lung, it is not able to act in the interstitium or alveolar cells and thus cannot act at the location of the initial pathology and further the location that is orchestrating the further pathology.

As a result, on traversing the alveolar-capillary membrane, inflammatory cells and fluid flood the alveoli. These interfere with surfactant function, which in turn reduces alveolar patency and compromises respiration. The ability of surfactant to protect the lung is further challenged with the initial destruction of surfactant-producing Type II pneumocytes during the disease process.

The present inventors have determined that pre- or peri treatment (prophylactic) using the lamellar compositions discussed herein as opposed to post treatment of fibrotic conditions can act to stop the fibrotic pathology before this becomes established and irreversible damage is done to the cell.

Radiation therapy (RT) is prescribed in over 50% of patients receiving cancer therapy. A potential serious complication is RILI, which affects normal lung within the radiation field. In particular, RT can cause pneumonitis and pulmonary fibrosis, following treatment to thoracic structures, chest wall and lower neck, either because the lung is part of the tissue being targeted by the RT or due to its proximity to the tumour target. RILI can occur due to accidental exposure or due to therapeutic treatment with ionizing radiation. Symptomatic radiation pneumonitis (pulmonary inflammation), is estimated to affect about 7% of all patients receiving RT to the chest, while over 40% of patients may show radiological changes indicative of lung injury.

RILI is particularly associated with radiation doses above about 2 Gray (Gy). In animal experiments, electron microscopic changes are seen in cells provided with such radiation within 1 hour of irradiation, with early release and depletion of surfactant, which is essential for maintaining alveolar patency. By 24 hours, intracellular surfactant-containing lamellar bodies are depleted. In susceptible individuals, these early changes become clinically evident about 4-12 weeks after a course of RT. Typical symptoms include shortness of breath, cough, and chest discomfort, with increased susceptibility to lung infections. Whilst this acute radiation pneumonitis ordinarily resolves with treatment, a proportion of patients will later experience symptoms relating to chronic progression of disease involving significant pulmonary fibrosis which can severely compromise lung function to a life-threatening extent.

RT risk is typically minimised by reducing the dose provided to the subject. However, new approaches to mitigate RILI, in particular fibrosis are required to improved outcomes in cancer survivors. Approaches to mitigate RILI would also be useful in the treatment of a deliberate or accidental nuclear or radiological event.

The inventors have determined that in subjects exposed to situations that may lead to compromised alveolar-capillary membranes, provision of synthetic lamellar body compositions, prior to, along with, or subsequent to an insult/injury which may lead to such compromised alveolar-capillary membranes, can limit the harmful progression of the initial injury towards alveolar fibrosis. In contrast to surfactant lipid containing compositions, the inventors have determined synthetic lamellar body lipid compositions that can act in the interstitium and alveolar cells.

In particular, the inventors have shown that with treatment with synthetic lamellar bodies of the invention fibrosis is reduced, as shown by a reduction in ASMA and collagen. Treatment with lamellar bodies of the invention suitably constitutes a novel treatment of aberrant fibrotic conditions including those in the lung, skin, GI, GU, heart, peritoneum, kidney, liver and other mucosal conditions subject to fibrosis. This treatment can be pre or peri treatment in relation to risk of as opposed to post treatment in relation to treatment of a defined condition (for example treatment provided before the pathology of fibrotic condition is observed). Using a Radiation Induced Lung Injury model in sheep the inventors have considered treatments to reduce fibrosis and myofibroblasts as measured by ASMA.

Myofibroblasts (MFB) are found in the subepithelial region of a range of mucosal tissues e.g. skin, gastrointestinal tract (GI) and genitourinary (GU) system. Myofibroblast regeneration and numbers increase in wound healing and tissue repair and on successful completion they are removed via apoptosis. However, in several fibrotic diseases e.g. in the liver, heart, lung, peritoneum and kidney failure of the regenerative process causes persistent myofibroblasts and promotes extracellular (interstitial) cell matrix (ECM) remodelling and growth Such remodelling and growth is a hallmark of fibrotic diseases. When this remodelling occurs, chronic fibrotic conditions occur which may be irreversible. Chronic fibrotic conditions typically require restorative as opposed to preventative treatments.

Myofibroblasts can arise from a number of progenitor cells, including cells of both endothelial and epithelial origin. Characteristically, myofibroblasts are marked by the presence of alpha-smooth muscle actin (ASMA or αSMA). In the context of the lung, myofibroblasts can also “arise de novo” directly from mesenchymal stem cells.

Further using the model system of fibrosis in the lung, the inventors have shown that the density of DC-LAMP positive cells was reduced in line with treatment with lamellar bodies of the invention, reducing profibrotic response in tissue injury.

It has been shown that the presence of dendritic cells is associated with interstitial fibrosis in a range of conditions e.g. kidney transplant rejection, interstitial lung diseases and psoriasis vulgaris. Furthermore, the density of myeloid dendritic cells during acute rejection could be an important risk factor for the long-term development of chronic changes and loss of graft function. Dendritic cell lysosome-associated membrane glycoprotein (DC-LAMP) is a marker for mature myeloid dendritic cell density. It is considered to be elevated in Non-Specific Interstitial Pneumonia. DC-LAMP expression is also increased in the skin in psoriasis vulgaris.

Accordingly, a first aspect of the present invention provides a method of treatment of fibrosis or reducing a profibrotic response, the method comprising the step of providing lamellar bodies of the invention to a subject in need thereof. Suitably the profibrotic response may be provided in the lung, skin, GI, GU, heart, peritoneum, kidney liver or other mucosa. Suitably a condition causing fibrosis may be provided in the lung, skin, GI, GU, heart, peritoneum, kidney liver and other mucosa. Suitably, there is provided prophylactic treatment of damage (risk of damage) caused by compromised alveolar-capillary membranes in a mammalian subject, comprising administering to the subject a lamellar body composition in a treatment effective amount prior to, along with, or subsequent to injury which may lead to compromised alveolar-capillary membranes.

Without wishing to be bound by theory, the inventors consider the provided lamellar body may aid alveolar opening through reducing surface tension, and further may beneficially modulate the nature and extent of pathophysiologic processes that underlay acute inflammatory and/or progressive fibrotic responses in the distal lung.

Suitably, following administration of the lamellar body composition, mitigation of potential damage to the subject caused by fibrosis or profibrosis can be determined by measuring DC-LAMP expression or ASMA. It is considered such mitigation is more than the lamellar bodies acting to merely replace depleted surfactant levels. This is supported by the prophylactic effects of the lamellar bodies discussed herein prior to radiation damage and thus prior to surfactant depletion. Without wishing to be bound by theory, it is considered the effects of the lamellar bodies of the invention are provided by it is ability to traverse the cell membrane (unlike surfactant). Suitably, following administration of the lamellar body composition, mitigation of potential damage to the subject caused by compromised alveolar-capillary membranes can be determined by measuring the resultant cellular and stromal response in subjects. In the present study a sheep model indicative of the human response to radiation-induced direct lung injury demonstrates that provision of the lamellar body composition abrogated radiation-induced alveolar fibrosis and increased the number of cells expressing a marker for surfactant-producing type II pneumocytes in the distal lung. It is further considered that this mitigation of fibrotic conditions allows the lamellar body compositions of the present invention to be used in the treatment of conditions such as Acute Lung Injury and Adult Respiratory Distress System (ARDS).

According to a second aspect of the present invention there is provided lamellar bodies for use in treating fibrosis or a profibrotic response. The treatment can be applied pre or peri in relation to risk (prophylactic treatment) rather than based on post treatment of a defined condition or based on damage observed due to fibrosis. Suitably the fibrosis or profibrotic response may be provided in the lung, skin, GI, GU, heart, peritoneum, kidney liver or other mucosa. Suitably the fibrosis or profibrotic response may be in lung and be due to an injury associated with compromised alveolar-capillary membranes in a mammalian subject. Suitably, the lamellar body composition may be provided prior to, along with, or subsequent to insult/injury associated with compromised alveolar-capillary membranes. As discussed above, in contrast to the lipid compositions of surfactant, it is considered the synthetic lamellar body compositions of the present invention can cross the alveolar cell wall and affect the initial and function-limiting pathology of the condition occurring in the alveolar interstitium (the thin zone of connective tissues within the walls of pulmonary alveoli) and within the alveolar cells.

According to a third aspect of the present invention there is provided a lamellar body composition formulated for administration via the airway to the epithelium of the lower airways for the prevention and treatment of distal lung injury due to compromised alveolar-capillary membranes, for example as caused by direct or indirect insults of the lung, including, but not limited to irradiation of the lower neck, thoracic structures or chest wall. As will be appreciated, suitably compositions may be provided to allow treatment of skin, GI, GU, heart, peritoneum, kidney liver or other mucosa.

According to a fourth aspect of the present invention there is provided a composition for use in the manufacture of a medicament for the treatment of pro-fibrotic conditions or fibrotic conditions, in particular conditions of the lung, skin, GI, GU, heart, peritoneum, kidney liver or other mucosa. The composition is a synthetic lamellar body composition with at least three lipids selected from phosphatidylcholine (PC), and cholesterol (Chol) and at least a further phospholipid selected from phosphatidylglycerol (PG), phosphatidylserine (PS) or phosphatidylinositol (PI) to provide a negatively charged phospholipid. Suitably the negatively charged lamellar body has a charge of about −30 mv or more negative. Suitably the lamellar body is sized such that it is at less than or equal to 250 nm, less than or equal to 200 nm, less than or equal to 150 nm, less than or equal to 125 nm, wherein the measurements relate to a diameter of the lamellar body—the lamellar body being considered to be substantially spherical.

Suitably the composition is for use in the manufacture of a medicament for the prevention and treatment of distal lung injury due to compromised alveolar-capillary membranes, for example as caused by direct or indirect insults of the lung, including, but not limited to irradiation of the lower neck, thoracic structures or chest wall.

Distal lung injury can result from direct lung injury, such as trauma, septic shock, or by irradiation. The latter can be provided by elective radiotherapy or alternatively irradiation may be due to accidental exposure to radiation. Chest irradiation can be via radiotherapy. Radiotherapy to the chest is most commonly given in cases of breast and lung cancer and in Hodgkin's disease.

For example, suitably irradiated lung injury may show deep red congestion evident on the pleural surface and firmness on palpation. Suitably the irradiated lung may show consistent histopathological features such as subpleural, periarteriolar and peribronchial intra-alveolar oedema, alveolar fibrosis, interstitial pneumonia and pneumocyte type II hyperplasia.

Suitably, treatment with the lamellar body composition may minimise or stop an increase in alveolar fibrosis. Suitably, treatment with the lamellar body composition may minimise or stop an increase in alpha-smooth muscle actin (ASMA) expression in the subject exposed to radiation. In particular, treatment with a lamellar body composition may abrogate injury-induced alveolar fibrosis and reduce ASMA expression.

Suitably treatment with the lamellar body composition may be associated with an increased number of dendritic cell-lysosomal associate membrane protein (DC-LAMP) +ve cells throughout the lung.

Without wishing to be bound by theory, the inventors further consider the lamellar bodies when provided to the lung, for example by nebulisation, can act on the pathological cascade leading to fibrotic conditions and provide additional lipid membranes to mitigate against fibrotic damage such as in RILI. Advantageously, methods to mitigate RILI would allow the development of more effective radiotherapy protocols.

With respect to RILI, aerosol delivery of a pulmonary protectant such as surfactant to the lung is considered to be advantageous as it provides for the use of smaller doses of pulmonary protectant, more rapid targeting of the tissues and thus minimises the side-effects that may occur. Aerosol delivery, for example by nebulising the lamellar body composition is also considered to allow ease and speed of provision of the lamellar body composition to the lung tissue for prevention or treatment of RILI. Suitably the lamellar body composition may be provided by non-medical personnel.

Based on the studies undertaken by the inventors, key structural and functional requirements of lamellar bodies to provide for inhibited fibrotic response have been determined. Advantageously the lamellar bodies of this invention may comprise at least one, and suitably a combination of the following features:
be anionic as opposed to cationic:
provide for intracellular and/or interstitial penetration, advantageously by provision of suitably sized, substantially spherical lamellar body structures that can enter cells and/or interstitium as opposed to simple non-spherical monolayered surfactants/PC.
the phospholipids of the lamellar bodies are not limited in their distribution to the cell membrane.

In contrast to previous work which suggested that phospholipids (e.g. PC) and/or surfactant may provide a restorative effect to fibrotic tissues i.e. provide alleviating effects without mitigating the causes of the damage or reversing the damage, the present inventors have determined that lamellar bodies can provide preventative effects by pre-treatment of cells of a subject with the lamellar bodies. This is significant as such preventative use can minimise the damage caused by a profibrotic response rather than merely provide a reduction in the effects of such damage.

The term “mammalian subject” is preferably a human. In embodiments the human can be a subject who has undergone lung injury or a subject undergoing radiation for cancer treatment, in particular for lung cancer treatment or cancers predominately related to smoking. In embodiments the subject is a human at risk of a fibrotic condition or with pro-fibrosis for example, cirrhosis, atrial fibrosis, endomycardial fibrosis, arthrofibrosis, mediastinal fibrosis, nephrogenic systemic fibrosis, retroperitoneal fibrosis, or sceleroderma/systemic sclerosis. In alternative embodiments, the invention provides for the veterinary treatment of non-human animals.

In embodiments, lung injury can be caused by for example trauma or ionizing radiation injury, for example an acute ionizing radiation injury e.g., caused by acute exposure to ionizing radiation over a time of less than 1 or 2 days or several days more.

In embodiments radiation exposure may not be associated with any clinically overt adverse effect. In embodiments, the administering step can be carried out pre- or post damage causing fibrosis, suitably within 1, 2, 3, or more days pre- or post damage that causes fibrosis. In embodiments, the administering step can be carried out within 1, 2, 3, or more days pre- or post lung injury, for example pre or post-ionizing lung injury or radiation exposure. Exposure can be elective, i.e. for radiation therapy of tumours or accidental i.e. industrial or military.

Suitably pre-treatment of the subject prior to exposure to RT, in particular lung radiotherapy is provided. Treatment can be via a nebulised lamellar composition.

Suitably, the treatment of pulmonary injury e.g. due to radiation may be by direct administration of the lamellar body composition to the epithelium of the lower airways by the inhalation of the composition or a nebulized solution of the composition or by any other means of direct administration to the lower airways.

Suitably the composition may be administered to the alveoli. Suitably the composition may be provided to the bronchioles.

Advantageously, the lamellar body composition may be delivered directly to the tissue for which treatment or protection from radiation is desired. Advantageously, the lamellar body composition may be administered immediately before or with each dose of radiotherapy, for example where radiotherapy is provided to the chest, and at various other times during and after a course of radiotherapy.

The invention is also suitable for use in imminent or recent radiation exposure due to an ionizing radiation event such as an attack or accident and for more prolonged exposure to background radiation following such an event or before an event where a patient is at risk for example of sepsis or aspiration of stomach contents. Thus the lamellar body composition may be provided as a protective treatment.

In the embodiments the lamellar composition can comprise at least three lipids selected from phosphatidylcholine (PC), and cholesterol (Chol) and at least a further phospholipid selected from phosphatidylglycerol (PG), phosphatidylserine (PS), and phosphatidylinositol (PI). In the embodiments the lamellar composition can comprise three lipids selected from phosphatidylcholine (PC), and cholesterol (Choi) and at least a further phospholipid selected from phosphatidylglycerol (PG), phosphatidylserine (PS), and phosphatidylinositol (PI).

Advantageously the third component, may be selected as PS over PG over PI.

In the embodiments the lamellar composition can comprise at least four lipids (components) selected from phosphatidylcholine (PC), and cholesterol (Choi) and at least two phosphoplipids selected from phosphatidylglycerol (PG), sphingomyelin (ESM), phosphatidyl ethanolamine (PE), phosphatidylserine (PS), and phosphatidylinositol (PI). The lamellar body should be provided such that it has a negative charge.

Based on work undertaken by the inventors it is considered at least one of PS, PI and PG should be provided in a combination of three or more (for example four or five) lipids (suitably comprising PC and Chol and anionic lipid) to facilitate cell entry by the lamellar body formulations/compositions.

Suitably combinations of the phospholipids that may be used in the present invention can comprise:

| Formulation/ | | |
|---|---|---|
| No | Lipid Composition | Mass Ratio |
| 1 | PC/SM/PE/PS/PI/Chol (LMS-611 | about 55/19/8/4/3/10 |
| | downsized to ca 125 nm) | |
| 2 | PC/SM/PE/PG/Chol | about 55/19/8/6/10 |
| 4 | PC/SM/PS/Chol | 61/19/8/10 |
| 8 | PC/SM/PE/PS/PI/Chol/LysoPC | 54/19/8/4/3/10/1 |
| 1a | PC/SM/PE/PS/PI/Chol (LMS-611 | 55.1/19.4/8.2/4.1/3.1/10.1 |
| | downsized to ca 125 nm) | |
| 2a | PC/SM/PE/PG/Chol | 55.3/19.4/8.2/6.8/10.1 |
| 4a | PC/SM/PS/Chol | 61.7/19.4/8.9/10.0 |
| 8a | PC/SM/PE/PS/PI/Chol/LysoPC | 54.5/19.2/8.1/4.0/3.1/10.0/1.1 |
| 1b | DOPC/ESM/DOPE/DOPS/HSPI/Chol | 55.1/19.4/8.2/4.1/3.1/10.1 |
| | (LMS-611 downsized to ca 125 nm) | |
| 2b | DOPC/ESM/DOPE/DOPG/Chol | 55.3/19.4/8.2/6.8/10.1 |
| 4b | DOPC/ESM/DOPS/Chol | 61.7/19.4/8.9/10.0 |
| 8b | DOPC/ESM/DOPE/DOPS/HSPI/Chol/LysoPC | 54.5/19.2/8.1/4.0/3.1/10.0/1.1 |

| | | | | Zeta |
|---|---|---|---|---|
| | | Identifica- | Size | Potential |
| Lipids | Ratio w/w | tion Label | (nm) | (mV) |
| DOPC/ESM/DOPE/DOPS/ | 54.8/19.3/8.1/4/3.1/10 | LMS-611 | 123 | −24 |
| HSPI/Chol | | (sized at | | |
| | | 125 nm) | | |
| DOPC/ESM/DOPE/DOPS/ | 54.9/19.4/8.2/4/2.9/10 | LMS_DOPS/DSPG | 126 | −24 |
| DSPG//Chol | | | | |
| DOPC/ESM/DOPS/Chol | 69.5/14.9/9.9/5 | 009 | 113 | −25 |
| DPPC/DOPE/DOPS/Chol | 26.5/32.3/29.3/11.2 | 010 | 131 | −58 |
| DOPC/DOPE/DOPS/Chol | 27.9/31.7/28.8/11 | 011 | 108 | −55 |
| DOPC/DOPS/Chol | 60.3/28.2/10.8 | 012 | 110 | −53 |

The inventors have determined that a composition

| | | |
|---|---|---|
| LMS-611 | DOPS/ESM/DOPE/DOPS/HSPI/ | 55.5/19.4/8.2/4.1/3.1/10.1 |
| | Chol (LMS-611) | |

which is not downsized to provide lamellar bodies of a mean size less than 250 nm, preferably less than 200 nm, preferably less than 150 nm with a narrow size distribution, for example which remain as, on average larger, polydisperse microvesicles as formed in the manufacture of the lamellar bodies do not effectively enter the interstitium and/or within the cells and cannot minimise the profibrotic response in the entered cell.

To elucidate cellular interaction, prepared formulations, of prepared lamellar body formulations, were labelled with 1,1′-dioctadecyl-3,3,3′,3′-tetramethylindocarbocyanine perchlorate (Dil), a lipophilic non exchangeable fluorescent lipid label. Cellular interactions were measured by flow cytometry using trypan blue, a quencher of extracellular fluorescence, to discriminate between cell association and internalisation of the vesicles. This is a standard technique as described earlier (Sahlin et al., 1983; Feldmann et al., 2017). All variants were tested in HeLa cells with some also tested in A549 cells. The selected cell lines were taken as model cell lines to illustrate that the lamellar body compositions are taken up by cells i.e cross cell walls, which allows them to play a role in the prevention of fibrotic conditions.

Cellular entry of lamellar body compositions/formulations was observed with compositions/formulations prepared from six, five, four or three lipids (including cholesterol), containing negatively charged lipids. The phospholipids in the formulations comprise esterified saturated and unsaturated fatty acids. The example illustrates that lamellar body formulations are suitable to be taken up in general by cells i.e cross cell walls

In embodiments the composition can comprise at least five lipids selected from cholesterol, phosphatidylcholine, phosphatidylglycerol, sphingomyelin, phosphatidyl ethanolamine, phosphatidylserine, phosphatidylinositol and provided the lamellar body composition provided has a negative charge. Suitably the lamellar bodies comprise phosphatidylcholine and cholesterol. Suitably the negative charge may be at least −30 mV. Suitably the lamellar body may be sized less than 250 nm, preferably less than 200 nm, preferably less than 150 nm, suitably at about 125 nm.

The lamellar body formulations are downsized using standard processing techniques such as extrusion, dual centrifugation and microfluidisation

Suitably a lamellar body of the inventions is anionic in charge as determined by any methodology to measure zeta potential of phospholipid vesicles.

It would be understood in the art which lipids are negatively charged and which lipids are neutral, and which are zwitterions (neutral at pH 7). The overall charge provided by a lamellar body is considered to be provided by the charges provided by the net charge of the individual lipids. Typically, the more PS, PI and/or PG providing in a lipid composition the more negatively charged a vesicle formed of a lipid composition is likely to be.

Suitably negatively charged phospholipids are provided to provide a negative charge of more negative than −30 mV (about 10% PS) as this advantageously gives improved cell entry and the lamellar body should be more stable.

Suitably phosphatidylcholine and/or phosphatidylglycerol may constitute about at least 25%, at least 35% at least 40% to 70% of the lamellar body composition. Suitably phosphatidyl ethanolamine, phosphatidylserine, phosphatidylinositol and cholesterol may each independently be provided at up to about 15% of the composition, suitably at up to about 10% of the composition. Suitably the cholesterol may be provided at least at about 5% of the composition.

Suitably phosphatidylserine may be provided at least 10%, 15%, 20%, 25% or at least 30% or higher in the composition.

Suitably the lamellar body composition comprises at a minimum phosphatidyl choline and cholesterol and anionically charged lipid, with phosphatidyl serine being determined to be particularly advantageous. In particular, the lamellar body composition comprises about 44-70% phosphatidylcholine and/or phosphatidylglycerol in combination, about 4-12% cholesterol by weight, and optionally about 15-23% sphingomyelin, about 6-10% phosphatidyl ethanolamine, about 2-6% phosphatidyl serine, about 2-4% phosphatidyl inositol. Suitably the lamellar body composition may comprise 25%-70% phosphatidylcholine and/or phosphatidylglycerol in combination, about 4-12% cholesterol by weight, optionally about 15-23% sphingomyelin, at least 10% phosphatidyl serine, and optionally about 6-10% phosphatidyl ethanolamine, about 2-4% phosphatidyl inositol to a total % wt of 100%. As would be understood whilst about 10% phosphatidyl serine provides suitable negative charge, other combinations of lipids may be used in combination with PS or as an alternative to PS to provide a negative charge.

Without wishing to be bound by theory, it is considered the bulk of the lamellar body can be provided with phosphatidyl choline—this is relatively economical constituent of the lamellar body. Cholesterol provides rigidity and endurance to the lamellar body. Sphingomyelin acts to support the function of cholesterol. The negatively charged phospholipid is surprisingly determined to allow the lamellar body to enter the cell and for the lamellar body to exert anti-fibrotic effects. Thus, based on this understanding of the components of the lamellar body suitable combinations of lipids may be used to achieve the functional effects that the lamellar bodies are anionic as opposed to cationic and provide for intracellular and/or interstitial penetration, advantageously such penetration can be provided by provision of suitably sized, substantially spherical lamellar body structures that can enter cells and/or interstitium as opposed to simple non-spherical monolayered surfactants/PC. As discussed, the charge and ability of the lamellar body to enter cells and/or interstitium can be determined using the methods herein.

Suitably the lamellar body composition may comprise about 44-70% phosphatidylcholine, about 15-23% sphingomyelin, about 6-10% phosphatidyl ethanolamine, about 2-6% phosphatidyl serine, about 2-4% phosphatidyl inositol and about 4-12% cholesterol by weight and further comprise about 0-3% by weight of lysophosphatidyl choline.

Suitably the lamellar body composition may comprise about 54% phosphatidylcholine, about 19% sphingomyelin, about 8% phosphatidyl ethanolamine, about 4% phosphatidyl serine, about 3% phosphatidyl inositol and about 10% cholesterol by weight. Suitably the lamellar body composition may further comprise about 2% by weight lysophosphatidyl choline.

Suitably the lamellar body composition may be nebulised. As will be understood in the art, the size of the ‘droplets’ provided by the nebuliser will determine where they are deposited in the lung. Suitably, each ‘droplet’ may contain multiple lamellar bodies if the droplet is large and the lamellar bodies are small. For alveolar deposition i.e. the most distal part of the lung, the droplet size is typically on average around 1.5 microns. For central deposition i.e. to hit the airways, typically the average droplet size should be around 3.5 microns. Suitably a polydisperse composition may be provided with a droplet size of about 5 microns if the lamellar bodies are not sized. Suitably the lamellar bodies may be provided in a droplet with an average size of about 100 nm (as would be understood such a size is in cross-section or diameter if the lamellar bodies are considered to be spherical). Suitably a droplet size may be provided to direct lamellar bodies to a particular location in the lung suitably in relation to the location of the tumour or lung insult. Alternatively, a range of droplet sizes may be provided, suitably with larger dose where required, to cause exposure of the whole lung to the lamellar bodies being provided.

Suitably instillation and/or nebulisation may be used as suitable routes of administration for alveolar delivery of the lamellar bodies.

To maximise alveolar delivery, droplets with lamellar bodies may themselves be provided with an average size about 1.5 microns. Suitably the lamellar body may have a mass median aerodynamic diameter (MMAD) of about 4 microns. Suitably MMAD may be determined in a next generation impactor.

Currently amifostine is the only drug approved by the U.S. Food and Drug Administration for protection from radiation. Administered as an inactive prodrug it is dephosphorylated by alkaline phosphatase in the normal endothelium to form an active thiol which scavenges free radicals, induces cellular anoxia and protects DNA. Although several non-randomized clinical trials have demonstrated that amifostine can reduce the severity of lung injury after radiotherapy, its use has been generally limited to head and neck cancer patients because of its sometimes severe side effects.

Suitably, the lamellar body composition may be provided in combination with another treatment of fibrosis wherein the second treatment is provided separately to the lamellar body. Suitably a treatment selected from amifostine, melatonin or an antioxidant analogue or metabolite, for example an antioxidant such as vitamin E, coenzyme Q10, alpha-lipoic acid or vitamin C as active substances or any suitable oxygen radical scavenger may be provided. Such combinations of treatment may be provided prophylactically to the cells to minimise the effects of fibrosis.

Administration to the airways may be by inhalation or by intratracheal, intrabronchial or bronchoalveolar administration.

Methods of intratracheal, intrabronchial or bronchoalveolar administration include, but are not limited to, spraying, lavage, inhalation, nasal insufflation, flushing or installation, using as fluid a physiologically acceptable composition in which the pharmaceutical composition has been dissolved. When used herein the terms “intratracheal, bronchial or alveolar administration” include all forms of such administration whereby the composition is applied into the trachea, the bronchi or the alveoli, whether by the instillation of a solution of the composition, by applying the composition in a powder form, or by allowing the composition to reach the relevant part of the airway by inhalation of the composition as an aerosolized or nebulized solution or suspension or inhaled powder, with or without added stabilizers or other excipients.

Methods of bronchial or alveolar administration also include bronchoalveolar lavage (BAL) according to methods well known to those skilled in the art, using as a lavage fluid a physiologically acceptable composition in which the composition has been dissolved, or by the direct application of the composition, in solution or suspension or powder form during bronchoscopy. Methods for intratracheal administration include blind tracheal washing with a similar solution of dissolved composition or with a suspension of the composition, or the inhalation of nebulized fluid droplets containing the dissolved composition or a suspension of the composition, obtained by use of any nebulizing apparatus adequate for this purpose.

In the context of protecting the lung, pulmonary delivery potentially allows the use of smaller doses that target more rapidly than systemic delivery of active agents and consequently avoids systemic side-effects. Further, dose, targeting and side-effect issues, administering pulmonary protection via aerosol offers many of the same advantages that are cited in the context of mass vaccination by the same route—namely ease and speed of application by nonmedical personnel, non-invasiveness resulting in greater social acceptance, reduced risk of cross-contamination of blood-born infectious agents, diminished medical waste, and potentially lower costs.

The present invention is primarily concerned with the treatment of human subjects, but the invention may also be carried out on animal subjects, particularly mammalian subjects such as dogs, cats, livestock and horses for veterinary purposes. Subjects may be male or female and may be of any age, including neonate, infant, juvenile, adolescent, adult, or geriatric subjects.

“Ionizing radiation” as used herein includes both electromagnetic radiation (such as X-ray radiation and gamma radiation) and particle radiation (including alpha, beta, neutron, and proton radiation). Ionizing radiation is characterized by carrying sufficient energy to ionize atoms and molecules: to generate positive or negative particles from electrical neutral atoms and molecules. When passing through matter, for instance a cell, tissue, or organism, the ionizing radiation discharges energy. When sufficiently high, this can lead to acute or chronic injury to the cell, tissue or organism.

“Treat” as used herein refers to any type of treatment that imparts a benefit to a patient, including delaying the onset and/or reducing the severity of at least one symptom of the disorder (for example, decreasing cell death, and/or treating one or more of leukopenia, neutropenia, monocytopenia, lymphocytopenia, fatigue, etc.).

The terms “a” and “an” as used herein refer to “one or more” of the enumerated components. It will be clear to one or ordinary skill in the art that the use of the singular includes the plural unless specifically stated otherwise.

As used herein, the term “effective amount” means that amount of a drug or pharmaceutical agent that will elicit the biological or medical response of the pulmonary tissue or of the animal that is being sought.

Pharmaceutical Formulations

Suitably, the lamellar body composition may be formulated for administration in a pharmaceutical carrier in accordance with known techniques. See, e.g., Remington, The Science And Practice of Pharmacy (9th Ed. 1995). In the manufacture of a pharmaceutical formulation according to the invention, the lamellar body composition is typically admixed with inter alia, an acceptable carrier. The carrier must, of course, be acceptable in the sense of being compatible with any other ingredients in the formulation and must not be deleterious to the patient.

Formulations of the present invention suitable for administration may comprise sterile aqueous and non-aqueous solutions of the lamellar body composition.

Solid or liquid particulate forms of the lamellar body composition prepared for practicing the present invention should include particles of respirable size: that is, particles of a size sufficiently small to pass through the mouth and larynx upon inhalation and into the bronchi and alveoli of the lungs. In general, particles ranging from about 1 to 10 microns in size are within the respirable range. Particles of non-respirable size that are included in the aerosol tend to be deposited in the throat and swallowed, and the quantity of non-respirable particles in the aerosol is preferably minimized.

Aerosols of liquid particles comprising the lamellar body composition may be produced by any suitable means, such as with a pressure-driven aerosol nebulizer or an ultrasonic nebulizer. See, for example U.S. Pat. No.4,501,729. Nebulizers are commercially available devices which transform solutions or suspensions of the active ingredient into a therapeutic aerosol mist either by means of acceleration of compressed gas, typically air or oxygen, through a narrow venturi orifice or by means of ultrasonic agitation. Suitable formulations for use in nebulizers consist of the active ingredient in a liquid carrier, the active ingredient comprising up to 40% w/w of the formulation, but preferably less than 20% w/w. The carrier is typically water or normal saline or phosphate-buffered saline (PBS) (and most preferably sterile, pyrogen-free water or normal saline or PBS) or a dilute aqueous alcoholic solution, preferably made isotonic but may be hypertonic with body fluids by the addition of, for example, sodium chloride. Optional additives include preservatives if the formulation is not made sterile, for example, methyl hydroxybenzoate, antioxidants, flavouring agents, volatile oils, buffering agents and surfactants.

Subjects that may be treated by the methods of the present invention are those who have undergone direct or indirect lung injury, been or may be exposed to any level of potentially damaging ionizing radiation. For example, the subjects may be those who have been or may be exposed to 50 or 100 rads; 0.5 or 1 Gray; or 500 to 100 milliSieverts of ionizing radiation, or more. It is generally believed that radiation injury is characterized by delayed onset of symptoms after exposure to the injuring radiation, so it will be understood that treatment may be administered while the injury is at an early or latent stage, as well as during manifest illness.

Preferred features and embodiments of each aspect of the invention are as for each of the other aspects mutatis mutandis unless context demands otherwise.

Each document, reference, patent application or patent cited in this text is expressly incorporated herein in their entirety by reference, which means it should be read and considered by the reader as part of this text. That the document, reference, patent application or patent cited in the text is not repeated in this text is merely for reasons of conciseness.

Reference to cited material or information contained in the text should not be understood as a concession that the material or information was part of the common general knowledge or was known in any country.

As used herein, the articles “a” and “an” refer to one or to more than one (for example to at least one) of the grammatical object of the article.

“About” shall generally mean an acceptable degree of error for the quantity measured given the nature or precision of the measurements.

Throughout the specification, unless the context demands otherwise, the terms ‘comprise’ or ‘include’, or variations such as ‘comprises’ or ‘comprising’, ‘includes’ or ‘including’ will be understood to imply the includes of a stated integer or group of integers, but not the exclusion of any other integer or group of integers.

Embodiments of the present invention will now be described with reference to the accompanying figures in which:

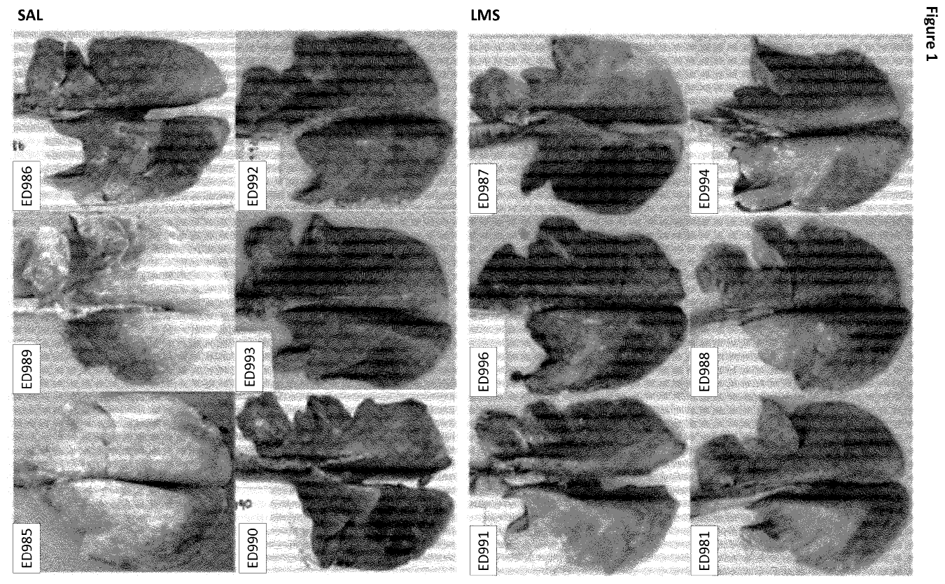

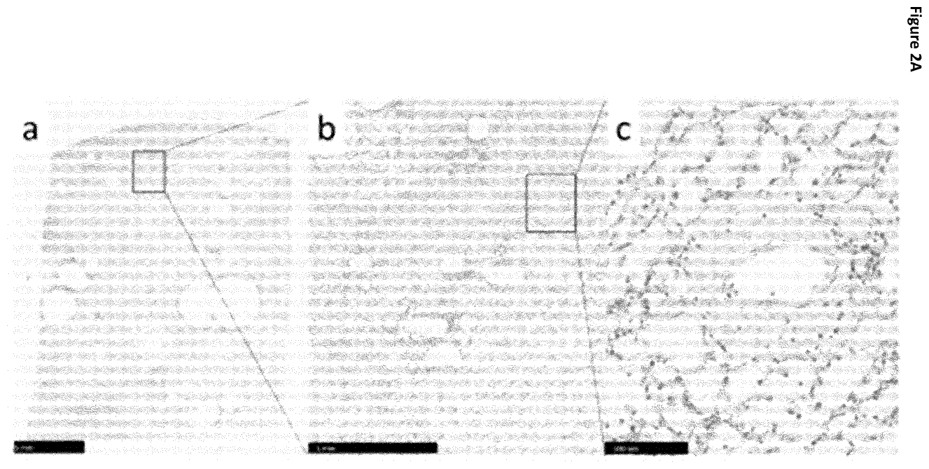

FIG. 1: illustrates the gross pathological features associated with lung injury, in this case with RILI in sheep—photographs are provided of lungs removed from sheep treated with nebulised saline (SAL) or Lamellar Bodies (LMS) prior to each of 5 fractions of 6 Gy radiation delivered to the left caudal diaphragmatic lobe at 3-4 day intervals. The area of dark red pleural discolouration which reflects the margins of the Planning Target Volume (PTV) was clearly evident in all the lungs. Whilst there was no substantial difference in the gross appearance of the lungs from each group there was clearly some sheep to sheep variation in the nature and extent of the radiation-induced discolouration.

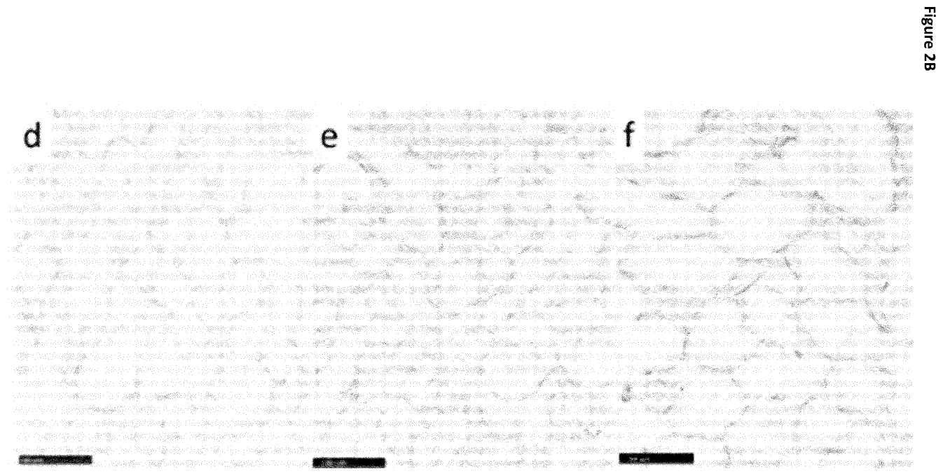

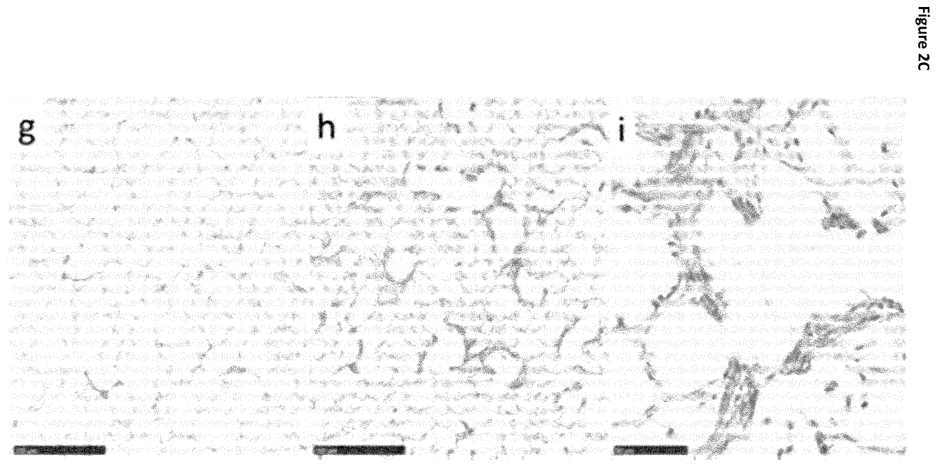

FIG. 2: illustrates histopathological features associated with lung injury, in this case RILI in sheep—Panels (a-c): provide photomicrographs of an H&E-stained section illustrating the oedema that arises as a consequence of radiation exposure to the sheep lung. Oedema could be appreciated macroscopically (a) and was frequently found in the subpleural region (scale bar 5 mm). The borders between areas of oedema and aerated lung were often sharply demarcated (b)(scale bar 1 mm). Perivascular oedema was also recognised (c) (scale bar 100 μm). Panels (d-f): photomicrographs of picrosirius red-stained sections highlighting collagen deposition. Panels (e) and (f) are section images from radioexposed lung (scale bars 100 and 50 pm respectively) and (d) from the contralateral control lung (scale bar 100 μm). Radiation exposure was associated with an increase in the area percentage of collagen. Panels (g-i): photomicrographs highlighting the expression of ASMA in radioexposed lung (h & i, scale bars 250 and 50 μm respectively), and from the contralateral control lung (g) (scale bar 250 μm). ASMA expression in the non-radioexposed lung was sparse and found in association with the alveolar ducts, both at the septal tips and in the alveolar walls. Radiation exposure led to an increase in ASMA expression in these areas.

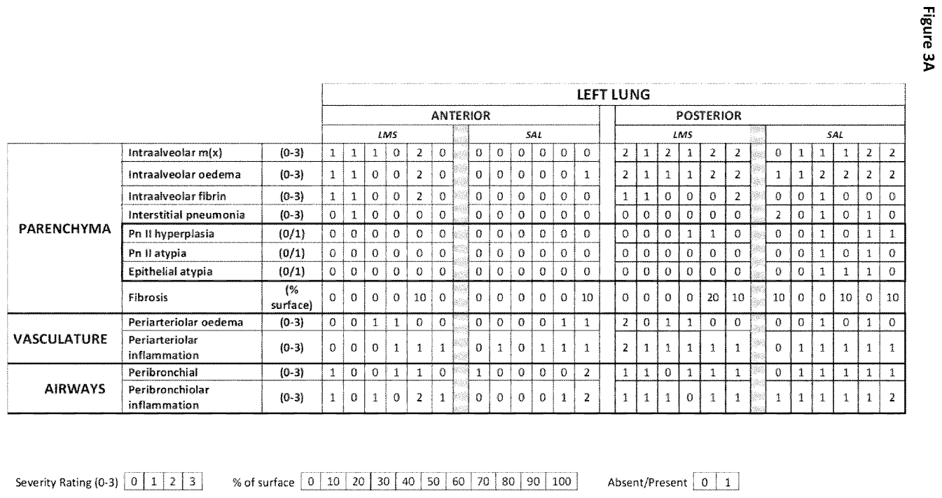

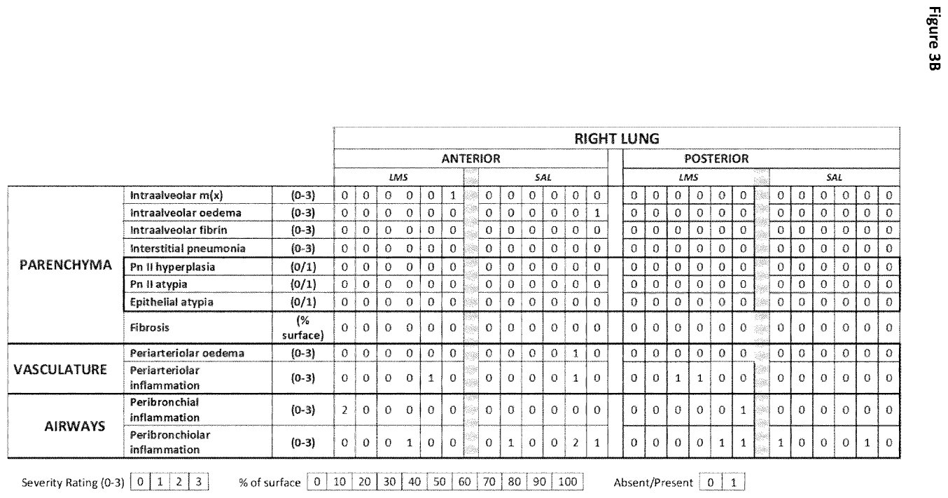

FIG. 3: illustrates histopathology quantitation wherein a heatmap representation of the results of blinded semiquantitative analysis is provided of the principal histopathological features associated with radiation exposure in the sheep lung. The upper panel reports the findings relating to the left lung, and the lower, the right lung. Within each lung the areas of assessment were further subdivided into posterior and anterior, reflecting the origin of the blocks submitted for assessment. Blocks derived from the posterior volume of the left lung lay within the PTV and were directly exposed to radiation, whilst blocks derived from the anterior volume lay proximal to the cranial margins of the PTV and should not have been directly exposed to radiation. Equally blocks from the right lung were identified as contralateral controls for the left lung samples. The panels are further subdivided according to the treatments, Lamellar Body (LMS) or saline (SAL), with the colour of each cell representing the semiquantitative scoring of the histopathological features listed to the left hand side of each row in the heatmap.

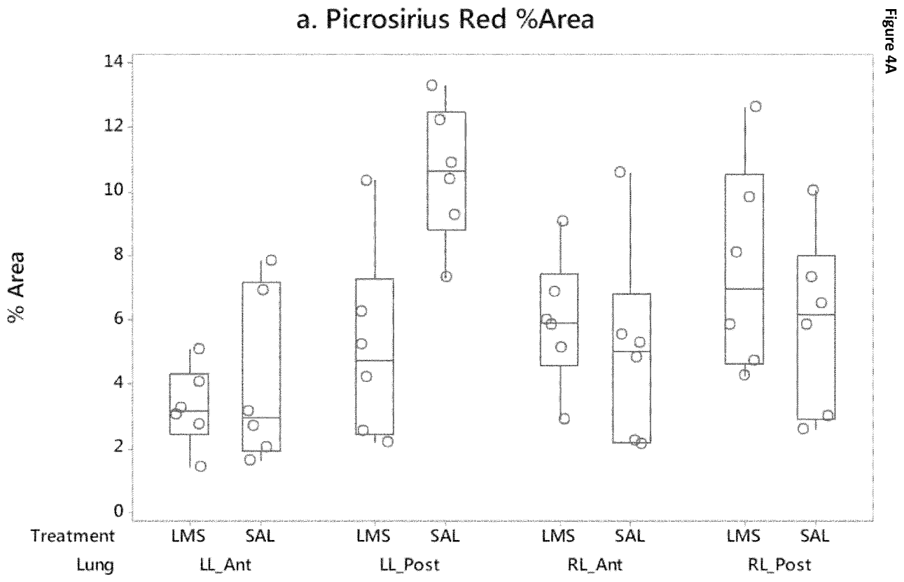

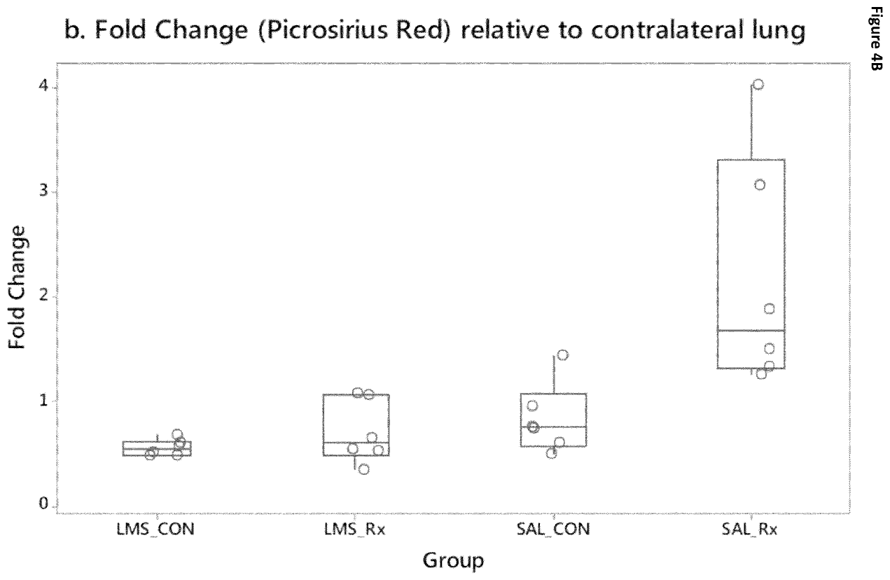

FIG. 4: illustrates Quantification of Picrosirius Red staining wherein (a) Boxplot depicting data relating to the percentage area of collagen present in Picrosirius red-stained lung parenchymal sections derived from the lungs of sheep previously exposed to radiation. Sections were derived from the left caudal diaphragmatic lung (LL_Post; representing the isocentre of the PTV), its contralateral control (RL_Post), a within-lung control sourced anterior to the cranial margin of the PTV (L_Ant), as well as its corresponding block from the right contralateral control lung (RL_Ant). Boxplots are further categorised according to the treatment (SAL or LMS) that the sheep received prior to radiation exposure. (b) Boxplot depicting the fold change in percentage area of collagen in sections derived from the left lung, relative to the right lung contralateral control sections paired within animal (LL/RL). LMS_CON and SAL_CON are the fold changes between LL_Ant and RL_Ant, and LMS_Rx and SAL_Rx are the fold changes between LL_Post and RL_Post. Only the sheep pre-treated with saline demonstrated a significant increase in fold change in the left (radioexposed) lung relative to the right contralateral control lung.

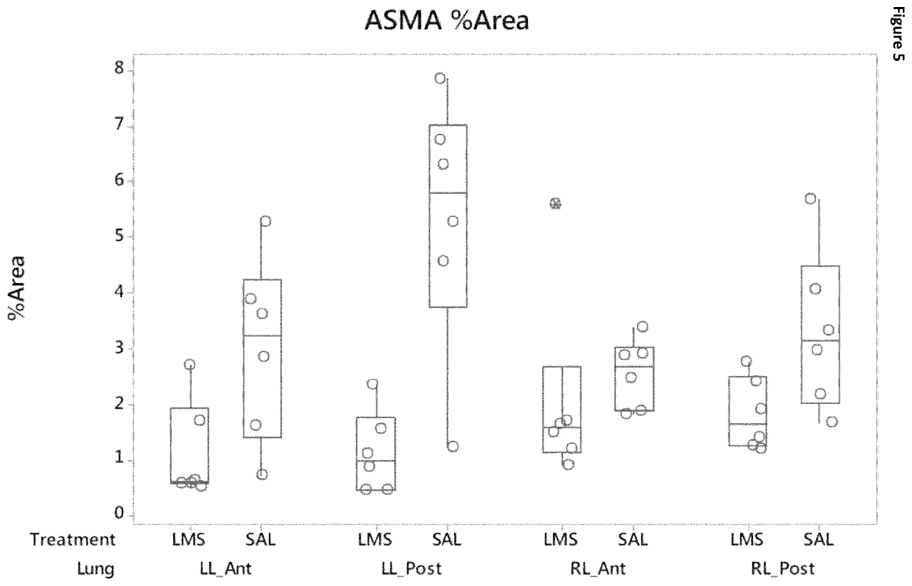

FIG. 5: illustrates the Quantification of ASMA staining—Boxplot depicting data relating to the percentage area of ASMA present in lung parenchymal sections derived from the lungs of sheep previously exposed to radiation. The source of the sections is as described in the legend for FIG. 4, and in the material and methods.

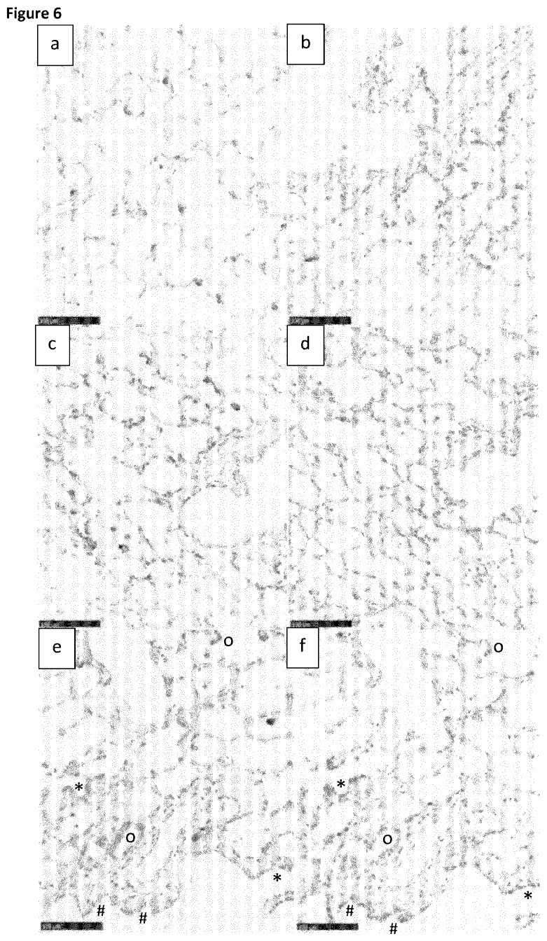

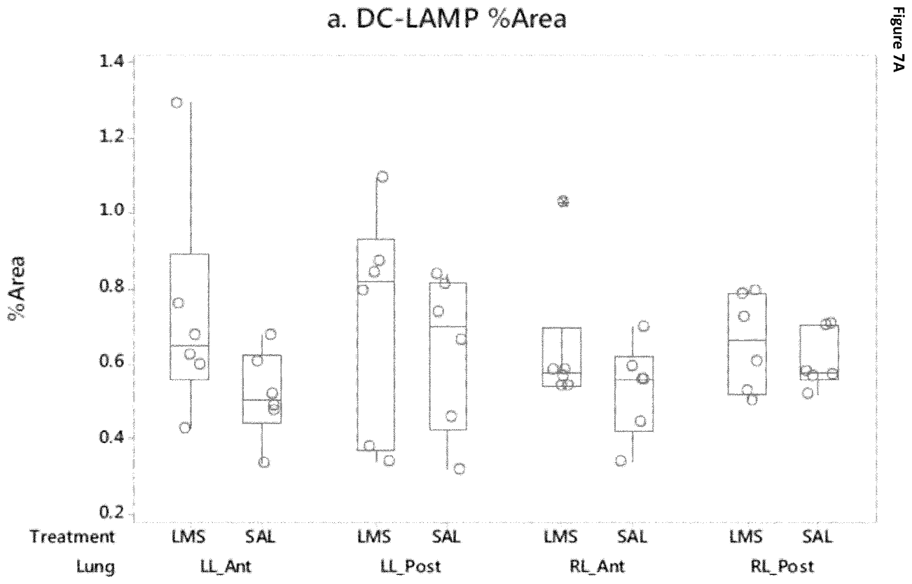

FIG. 6: illustrates DC-LAMP and Antigen Ki-67 (Ki-67—a marker for cell proliferation) expression associated with RILI in sheep wherein panels (a) and (b) are photomicrographs of sections immunostained to depict DC-LAMP expression from radio-exposed (b) and non-radio-exposed (a) contralateral control lung (scale bar 100 μm). In control lung DC-LAMP was expressed by rounded cells in the alveolar corners assumed to be type II pneumocytes. These cells were evenly spaced throughout the distal lung parenchyma. In radio-exposed lung clusters of DC-LAMP-expressing cells could be clearly identified lining the walls of alveoli, with a concomitant reduction in expression in the alveolar region (not shown). Where clusters of DC-LAMP expressing cells could be identified (c), we assessed whether these cells were proliferating by immunostaining neighbouring serial sections (d) to depict the expression of Ki67 (scale bars 100 μm). In many instances the clusters of DC-LAMP expression were not associated with obvious cell proliferation. Indeed, the photomicrographs from serial sections in panels (e) depicting DC-LAMP expression (scale bar 100 μm), and (f) depicting Ki67 expression (scale bar 100 μm) illustrate the sometimes concordance between DC-LAMP and Ki67 expression (*), and the fact that DC-LAMP expression can occur in the absence of Ki67 expression (o), and vice versa (#).

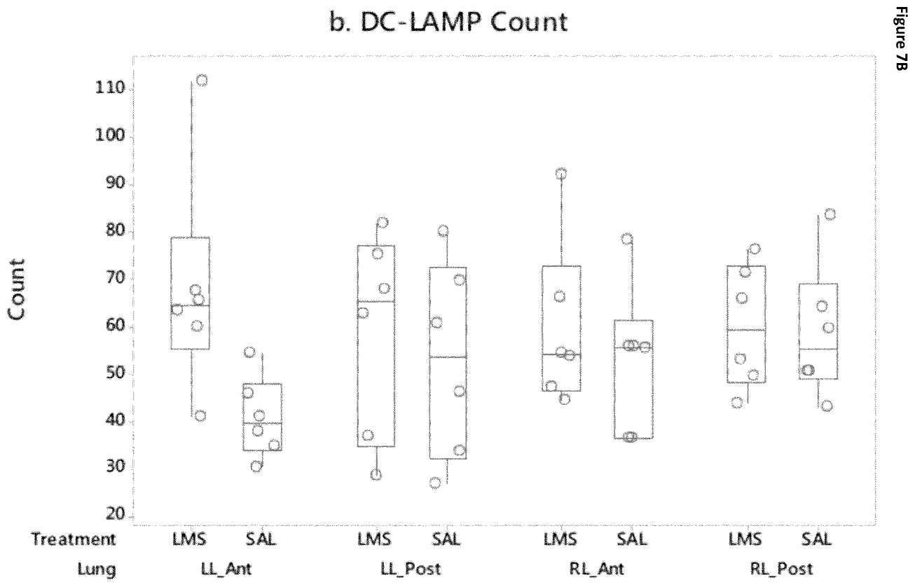

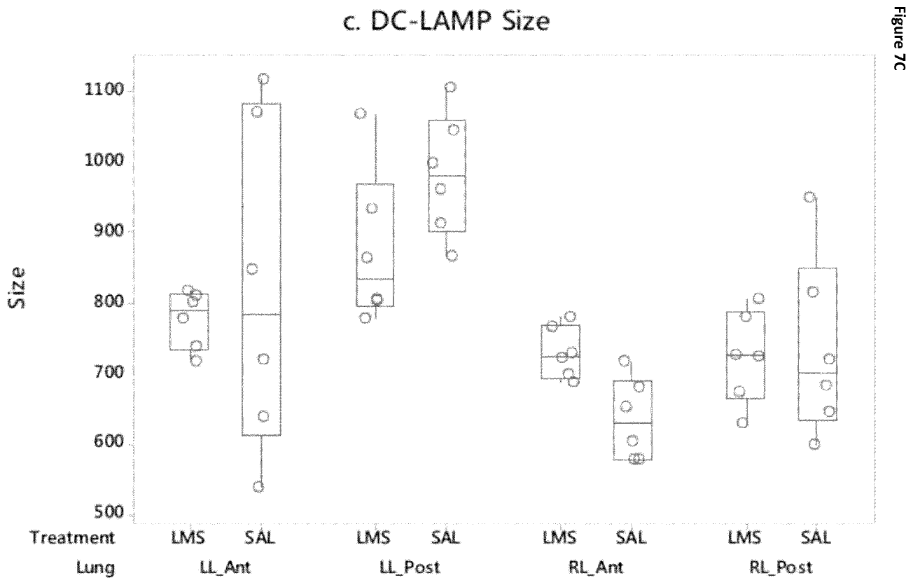

FIG. 7: illustrates Quantification of DC-LAMP staining wherein a boxplot is provided depicting data relating to (a) the percentage area of DC-LAMP expression, (b) the count of DC-LAMP expressing particles (DC-LAMP Count), (c) the average size of DC-LAMP-expressing particles (DC-LAMP size), and (d) the median Nearest Neighbour Distance (NND) as applied to DC-LAMP expressing cells present in parenchymal sections derived from the lungs of sheep previously exposed to radiation. The source of the sections is as described in the legend for FIG. 4, and in the material and methods.

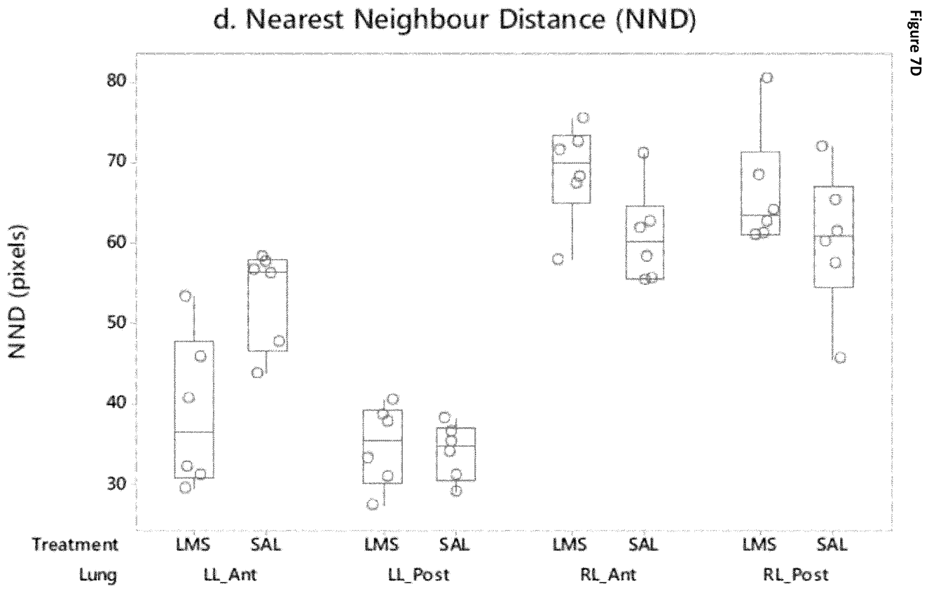

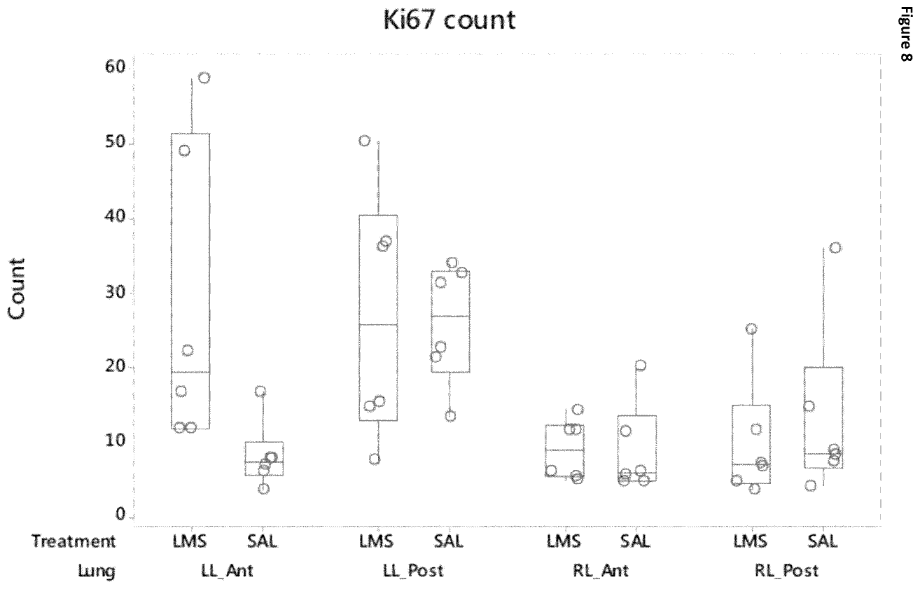

FIG. 8: illustrates Quantification of Ki67 staining provided by a boxplot depicting data relating to the average number of Ki67-expressing cells (Ki67 count) present in image fields of lung parenchymal sections sourced from sheep previously exposed to radiation. The source of the sections is as described in the legend for FIG. 4, and in the material and methods.

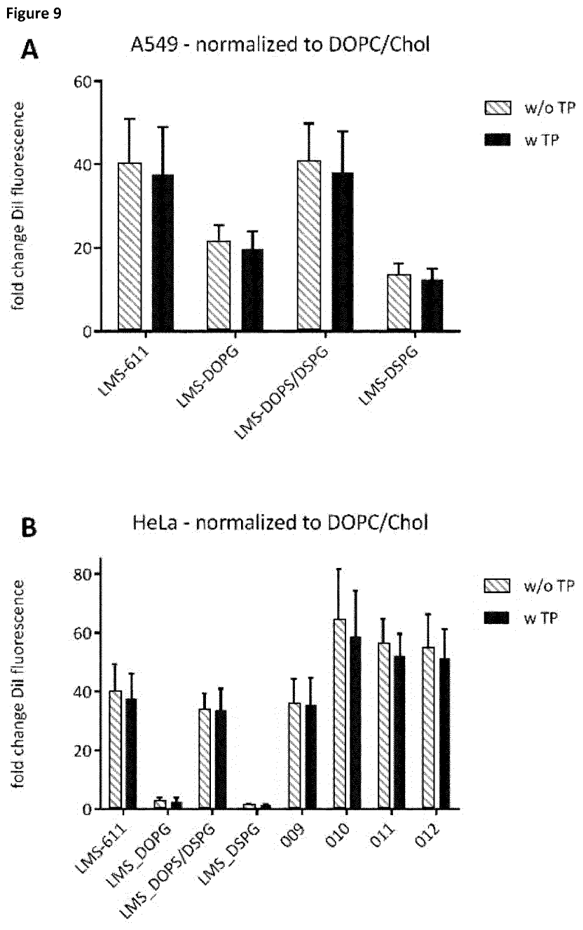

FIG. 9: illustrates total cellular association and internalisation of lamellar body formulations in A549 cells (A) and HeLa cells (B) after 2 hours of incubation. Median of Dil fluorescence was normalised to median of Dil of DOPC/Chol treated cells. Red bars (left hand side bars for each composition) represent total Dil fluorescence (i.e total of associated vesicles associated (internalized and adsorbed to the surface of the cells), blue bars (right hand side bars of each composition) represent fluorescence reduction after trypan blue quenching (i.e. internalized by the cells). Results are shown as mean with SD (n=2) apart from LMS-611 which was n=3.

### DETAILED DESCRIPTION

In the present description three model systems are described:
a Radiation-Induced Lung Injury (RILI) model;
a TGF-β1 model system used to consider profibrotic mediators and methods of screening of candidates to counter such profibrotic mediators. As discussed further below, expression of TGF-β1 has been found to be associated with lung fibrosis six months after radiation exposure. Thus, TGF-β1 expression was considered to be an indicative measure of the efficacy of candidates to mediate and reduce fibrosis or profibrotic mediators.
a cell entry model.

The RILI model was selected as a validated model of human pulmonary interstitial fibrotic response to damage. As would be known in the art, this model allows evaluation of the molecular mechanisms associated with radiation-induced lung injury and efficacy screening of candidate countermeasures.

Further this model allows the study of the development of fibrosis, and further recognised conditions of interstitial lung disease which cause effective disruption of the alveolar capillary membrane e.g. mild pneumonitis or pleural effusions.

### RILI Model

To provide a model of lung injury, twelve commercially sourced adult Shetland sheep (bodyweight: 38.5 kg [33.0-43.0] median [range]; 6 female and 6 castrated male) were included in the described study. Identification of animals was by means of ear tags. Animals were housed for the duration of the study and otherwise maintained according to normal standards of farm animal husbandry. The sheep were treated with anthelminthic before the study began. The sheep were randomly allocated to one of two sex-matched treatment groups.

In order to confirm the absence of pre-existing pulmonary disease and collect baseline samples against which to judge change within animals, preliminary baseline examination (BBr1) involving bronchoscopic visualisation, bronchoalveolar lavage and bronchial brush biopsy under gaseous anaesthesia was conducted. Where bronchoalveolar lavage cytology failed to meet normal boundaries and was indicative of parasitism (% eosinophils>7.5%) the sheep were re-treated with anthelminthic and results confirmed within normal range prior to any further involvement in the experimental protocols. A further two baseline examinations (BBr2 & BBr3) involving bronchial brush biopsy sampling were thereafter conducted at fortnightly intervals. Measurements of bodyweight and rectal temperature were also made at these time points. At least two weeks after the last baseline assessment (BBr3) the sheep were re-anaesthetised and positioned in sternal recumbency in order to facilitate the acquisition of thoracic computed tomography images for subsequent radiation treatment planning. Following the last radiation treatment (t0) the sheep were closely monitored for any evidence of adverse effect. At t0+11d and t0+21d the sheep were re-anaesthetised and subject to bronchial brush biopsy in the same manner as during the preliminary baseline evaluation. At t0+23d the sheep were killed by overdose of anaesthetic and presented for necropsy examination.

During radiation treatment a total dose of 30 Gy was delivered in a fractionated regime. This involved the delivery of 6 Gy to a defined planning target volume (PTV) of the left caudal diaphragmatic lung lobe on each of five separate occasions over a period of two weeks (3-4 days intervals).

Bronchoalveolar Lavage Collection

The bronchoscope (Model FG-15W; Pentax UK Ltd.) was wedged in the segmental bronchus of the right apical lobe. Two 20 ml aliquots of PBS were used to collect bronchoalveolar lavage fluid (BALF) from this lung segment. BALF samples were placed into sterile tubes and kept on ice until subsequent analysis. Five millilitres of BALF was removed and centrifuged at 400 g for seven minutes to separate out the cellular fraction. The resultant pellet was re-suspended in sterile phosphate buffered saline (PBS) and the total cell number counted before subsequent preparation of cytospins for differential cytology. Cells were counted using a Neubauer haemocytometer and values expressed per millilitre BALF. Cyto-centrifuge slides were prepared and stained using Leishman stain for differential counts on 500 cells. Cells were classified as neutrophils, macrophages, eosinophils, lymphocytes or mast cells according to standard morphological criteria.

Bronchial Brush Biopsy

On each occasion of baseline assessment three bronchial brush biopsy samples were derived from each of three separate areas of the lung (n=9 total). Samples were derived from bronchi within the left caudal diaphragmatic lung lobe (LCD), the right caudal diaphragmatic lung lobe (RCD), and also from bronchi within areas of the anterior right lung. On each occasion considerable care was taken (through manual mapping and reference to video recordings) to avoid sampling any area of bronchial epithelium that had previously been subject to bronchial brush biopsy. At t0+11d and t0+21d the sheep were subject to bronchial brush biopsy in the same manner as during the preliminary baseline evaluations.

Necropsy

Following euthanasia by intravenous injection of barbiturate, the heart and lungs were carefully removed from the carcase following standard necropsy protocols. The pulmonary circulation was perfused via the pulmonary artery with 2-3 litres of PBS before the heart was dissected away. The lungs were then photographed before being presented for further processing.

Inflation Fixation

Lung tissue was fixed by airway instillation of 10% neutral buffered formalin. The trachea was connected to a reservoir of fixative and the fixative allowed to flow until the ‘natural contours’ of the lung were established. The lungs were then floated in a tank of the same fixative and inflation-fixed at a constant pressure of 3.0 kPa for a period of 7 days.

Gross Tissue Sampling

After fixation each lung was carefully sliced along the transverse plane, starting at the caudal pole of each diaphragmatic lobe, into fifteen 1 cm thick tissue slices. These slices were then arranged in consecutive order for photographing prior to a representative tissue block from each contiguous slice being selected and carefully dissected from surrounding lung tissue. A further photographic image of the slices with their selected blocks in situ was captured to document the spatial origin of each block. This latter step was a necessary prerequisite to registering the position of each block with respect to the radiation field through reference to CT images previously collected from the same animals. Tissue blocks were then submitted for standard histological processing and paraffin embedding.

Block Selection

A formalin-fixed paraffin embedded (FFPE) tissue block from the left caudal diaphragmatic lung that represented the isocentre of the planning target volume was identified and selected, as was the corresponding block from the right contralateral control lung. These blocks were thenceforth labelled LL_Post and RL_Post respectively (“LL” and “RL” for left and right lung respectively, and “Post” for posterior). A further FFPE tissue block was sourced anterior (14.5 mm [9.7-23.0]) to the cranial margin of the PTV (LL_Ant), as well as its corresponding block from the right contralateral control lung (RL_Ant).

Histochemical and Immunohistochemical Staining

Sections cut from the above blocks were stained with haematoxylin-eosin (H&E) and Picrosirius Red, as well as being immunostained with antibodies specific for the following antigens—ASMA, DC-LAMP, and Ki67 protein. All slides were stained using standard immunohistochemistry methods with endogenous peroxidase blocked using 3% H₂O₂ in methanol and heat-induced antigen retrieval performed using 10 mM citrate buffer pH6.0.

ASMA—Non-specific binding was blocked with 10% Normal Goat Serum (Sigma G9023) in PBS+0.5% Tween 80. Primary antibodies Monoclonal ASMA (Sigma A2547) and Normal Mouse IgG isotype control (Sigma M5284) were diluted to 1 μg/ml in blocking buffer and incubated for 30 minutes at room temperature. Detection using biotinylated goat anti mouse IgG (Vector BA-2001) and Streptavidin peroxidase polymer (Sigma S-2438) followed by DAB substrate (Vector SK-4100) with Haematoxylin counterstain.

Ki67—Non-specific binding was blocked with 3% BSA (Sigma A3733) in PBS+0.05% Tween 20. Primary antibodies were Monoclonal anti Ki67 clone MIB-1 (Dako M7240) and Normal Mouse IgG isotype control (Sigma M5284) were diluted to 1 μg/ml for 45 minutes at room temperature. Detection using biotinylated goat anti mouse IgG (Vector BA2001) and Streptavidin peroxidase polymer (Sigma S-2438) followed by DAB substrate (Vector SK-4100) and Haematoxylin counterstain.

DC-LAMP Non-specific binding was blocked with 4% normal rabbit serum (Sigma R9133) in PBS+0.2% Tween 80. Primary antibodies DC-LAMP/CD208 (2BScientific DDX0191P-50) and Normal Rat IgG isotype control (Serotec MCA1125R) were diluted to 2.5 μg/ml in blocking buffer and incubated overnight at 4° C. Detection using biotinylated goat anti rat IgG (Vector BA4001) and Streptavidin peroxidase polymer (Sigma S-2438) followed by DAB substrate (Vector SK-4100) and Haematoxylin counterstain.

Bronchial Brush Cytokine Expression

Bronchial brush biopsy specimens were collected using cytology Brushes (Conmed Endoscopic Technologies 152R) agitated into 1 ml of cold sterile PBS (Sigma D8537) through 200 μl wide orifice pipette tips (Star Lab E1011-8000) and centrifuged at 10,000 g for 5 minutes. Pellets were resuspended in RLT buffer (Qiagen 74106) containing 1% β mercaptoethanol and stored at −80° C. until extraction. All samples were run through Qiashredder columns (Qiagen 79656) and RNA extractions were done using RNeasy mini kit (Qiagen 74106) with DNase treatment using Rnase free DNase set (Qiagen 79254). RNA was quantified on Nanodrop and quality checked on Agilent Tapestation with RNA screentape (Agilent 5067-5576). cDNA was prepared from 400 ng RNA with Transcriptor First Strand cDNA Synthesis kit (Roche 04 896 866 001) using random hexamer primers. Quantitative Real Time PCR was performed using Lightcycler 480 with 2.5 μl cDNA in LightCycler 480 Sybr Green I Master (Roche 04 887 352 001) and specific primers. Advanced relative quantification was calculated using Lightcycler 480 SW1.5 programme. Standard curves for each gene generated from pooled ovine alveolar macrophage cDNA. Melt curve analysis showed single peak for all samples. PCR efficiency was in range of 1.8 to 2.1. qPCR conditions and referenced primer sets (12-15) are stipulated in Tables 1_**qPCR conditions** and 2_**qPCR primer sets**.

Semiquantitative Histopathological Evaluation

One pathologist (SHS) examined a reduced subset (saline treated only) to obtain preliminary histopathology results to inform further analysis, whereas the other pathologist (JDP) was blinded to the results of the preliminary analysis and to the source of the slides. All H&E stained sections were scanned on a whole slide scanner (Nanozoomer, Hamamatsu, Japan) to acquire whole slide images (WSI) at ×40 magnification. These sections were then subject to detailed examination by a veterinary pathologist (Dr del-Pozo) blinded to their specific identity. Following an initial appraisal in which principal pathologic features were identified a semi-quantitative scoring system was developed to capture the incidence and extent of each feature amongst the different sections. Briefly, to score lesions for severity each section was allocated a score ranging from 0 (absent), 1 (mild), 2 (moderate), to 3 (severe). Fibrosis was scored by allocation of an estimated % surface involved (note that this score does not consider severity, which was mild in all cases in areas affected). Three variables, pneumocyte type II cell hyperplasia and atypia, and epithelial atypia, were scored qualitatively i.e. presence or absence.

Quantitative Histological Analyses

Areas of alveolar oedema could be clearly identified and annotated in Masson's trichrome stained sections using the Hamamatsu NDP.view2 viewer software. The area of each tissue section was outlined by using the freehand region tool, as was the area occupied by large (cartilaginous) airways and associated blood vessels. Finally, the area of each section occupied by alveolar oedema was also annotated. Measured annotations were saved to file, and the percentage of ‘parenchyma’ occupied by oedema (% Oedema Area_{[parenchyma]}=(total area occupied by alveolar oedema/(whole section area minus large airways and blood vessels))*100) was calculated.

Within ImageJ, the NDPITools custom extract to TIFF/mosaic plugin was used to extract each ndpi image file to multiple TIFF images. WSI from H&E stained sections were extracted at ×20 resolution, and the remaining WSIs at ×40 resolution. Image fields containing parenchyma (including airways no larger than respiratory bronchioles) were then manually selected from a random selection of these extracted files. These files were then converted to OME-TIFF using an ImageJ recursiveTiffConvert macro to engage the Bio-Formats exporter function.

Fractal analysis was applied to H&E-stained sections in order to assess the morphometry of the distal lung. Where available, 100 H&E-stained images were randomly selected from each section (LL_Ant, LL_Post, RL_Ant, and RL_Post). In the five instances where less than 100 images were available, 92, 78, 49, 90 and 56 images were selected. Parenchymal images were then converted to OME-TIFF using a recursiveTiffConvert macro to engage the Bio-Formats exporter function. In a manner similar to that described by Andersen et al (2012) these converted files were then processed to binary images using imageJ functionality and each binary image was analysed using the ImageJ FracLac plugin which calculates the fractal box dimension (D_{B}) (16).

The parenchymal ×40 OME-TIFF files were batch processed using macros employing the colour devolution plugin for detecting the area of red-stained collagen in Picrosirius Red-stained sections, the area of diaminobenzidine (DAB) stain in ASMA and DC-LAMP immunostained sections, and the number of DAB-stained particles (>200 pixels²) in Ki67 immunostained sections. Sample size was considered acceptable if the standard error for percentage area measurement fell below 5% of the mean value for that measurement. In the six sections where this condition was not met, the standard error ranged from 5.0 to 7.3% of the mean. As the scarcity of Ki67-stained cells in control lung sections meant that the 5% limit could not be achieved for the majority of sections a pragmatic decision on sampling was taken. Between 172 and 198 fields on each Ki67-stained section were examined.

Statistical Analyses

Data was initially assessed for normality of distribution using a Kolmogorov-Smirnoff test. Where necessary data transformation was applied to normalise the distribution, and where such transformation failed, a rank-order transformation was applied prior to subsequent evaluation. For repeated measures data a General Linear Model was fitted in which responses in radio-exposed and contralateral control lung segments were evaluated with respect to time, and the experimental treatment (lamellar body composition (LMS), SAL). Sheep identity, nested within treatment, was considered a random factor in the design.

For the analysis of histopathological and immunohistochemical data a General Linear Model two-way analysis of variance was conducted on the influence of the two independent variables (Lung, Treatment) on the variable in question. Lung included four levels (LL_Ant, LL_Post, RL_Ant, RL_Post), and Treatment two levels (Lamellar body composition, SAL).

Results

No adverse effects were noted either as a consequence of aerosol delivery, or in relation to exposure to radiation. At every time point sheep were weighed and rectal temperature recorded.

Bodyweight data was rank transformed and analyzed by two-way ANOVA with repeated measures in one factor. Treatment (LMS, SAL) was statistically significant at the 0.05 significance level. The main effect for Treatment yielded an F ratio of F(1, 82)=6.32, p=0.031. The main effect for Time yielded an F ratio of F(9, 82)=1.58, p=0.136, indicating no significant effect with respect to bodyweight. The interaction Treatment*Time was significant F(9,82)=25.10, p=0.001, highlighting the increase in weight of only the LMS-treated sheep from Rx1 onwards.

Temperature data was similarly rank transformed and analyzed by two-way ANOVA with repeated measures in one factor. Neither treatment (LMS, SAL) nor Time were statistically significant at the 0.05 significance level. The main effect for Treatment yielded an F ratio of F(1, 82)=2.09, p=0.178, and the main effect for Time yielded an F ratio of F(9, 82)=1.19, p=0.313—both indicating no significant effect with respect to body temperature. The interaction Treatment*Time was significant F(9,82)=3.31, p=0.002, highlighting the increase in temperature of only the saline-treated sheep from baseline 2 & 3.

Whilst body temperature remained within normal limits at all times during the experimental protocol, there was a small but significant increase seen at baseline 2 and 3. As this may have been indicative of a subclinical phenomenon data from these time points were discarded. Data obtained at the first baseline evaluation was used instead as the selected baseline time point.

From the Bronchial brush biopsy cytokine expression, gene expression levels of IL1 beta, TGF beta and IL8 relative to ATPase were log₁₀-, rank-, and log₁₀-transformed respectively to normalise data distribution prior to two-way ANOVA with repeated measures in one factor.

Neither Treatment nor Time had a significant effect on Log10 IL1beta or rank-transformed TGFbeta expression levels in samples derived from RCD or LCD and there was no significant interaction between these terms. Similarly, for samples derived from RCD neither Treatment nor Time had a significant effect on Log10 IL8 expression levels and there was no significant interaction between these terms. However, for samples derived from LCD, although Treatment had no significant effect, Time did have a significant effect on Log10 IL8 expression levels (p=0.030)—reflecting a decrease in expression at Time point 4. There was no significant interaction between these terms.

At necropsy, the pleural surface covering the planning target volume was easily identifiable as a consequence of dark red discolouration (FIG. 1). The underlying lung substance felt firmer on palpation, and when investigated in one instance, the affected lung volume failed to inflate properly when connected to a large volume-calibration syringe.

From the histopathological evaluation, the main parenchymal abnormalities noted in all radiation treated lungs were subpleural, periarteriolar and peribronchial intraalveolar oedema (FIG. 2) characterized by periarteriolar and intraalveolar accumulation of homogeneous, proteinaceous material, with occasional fibrillar material (fibrin), and aggregates of eosinophilic smudged material (fibrin). This change was associated with increase in the number of intraalveolar macrophages, which featured foamy cytoplasm in these areas. In addition, there was evidence of alveolar fibrosis characterized by mild thickening of alveolar walls by deposition of pale eosinophilic fibrillar material, interstitial pneumonia involving infiltration of alveolar walls with small numbers of lymphocytes and plasma cells, scattered pneumocyte type II hyperplasia and occasional atypia, with increased nuclear:cytoplasmic ratio, apical blebbing, mild pleomorphism, and nuclei with finely stippled chromatin and small nucleoli.

Radiation-induced abnormalities associated with the airways included mild submucosal infiltration by lymphocytes and plasma cells, and bronchial and bronchiolar epithelial atypia similar to that described for pneumocyte type II cells. Other histopathological abnormalities noted in a small number of sections involved parasite granulomas which were interpreted as unrelated to the treatment.

The results of the histopathological assessment are depicted in FIG. 3. Statistical analysis of semiquantitative and qualitative aspects of the histopathological assessment involved ranking the ordinal data and subjecting the ranked data, categorised according to group (LMS, SAL), lung (LL, RL) and segment (Ant, Post), to one-way ANOVA. Tukey pairwise comparisons indicated that for sheep treated with saline significantly increased features in radio-exposed lung relative to unexposed control lung of the same sheep included the number of intra-alveolar macrophages, the extent of alveolar oedema, the extent of interstitial pneumonia and pneumocyte type II hyperplasia, as well as the extent of peribronchial and periarterial inflammation. Pre-treatment with the lamellar body composition of the invention significantly mitigated the extent of radiation-induced interstitial pneumonia.

Results of Quantitative Histochemistry and Immunohistochemistry

Evidence of histopathological abnormality including oedema in the distal lung parenchyma raised the possibility that local lung compliance and hence morphometry of the alveolar region would be affected. Fractal analysis was applied in this context. As indicated by Porzionato A, Guidolin D, Macchi V, Sarasin G, Grisafi D, Tortorella C, et al. Fractal analysis of alveolarization in hyperoxia-induced rat models of bronchopulmonary dysplasia. Am J Physiol Lung Cell Mol Physiol. 2016; 310(7):L680-8. the fractal dimension, which measures the rate of addition of structural detail with increasing magnification, scale, or resolution, can be used to characterize the spatial pattern formed by the alveolar walls.

H&E stained slides were scanned to digital images and the NDPITools custom extract to TIFF/mosaic plugin was used to extract each .ndpi image file to multiple ×20 TIFF images. Non-parenchymal images were manually deleted and a random selection of these files was made. Where available, 100 images were randomly selected—if less than 100 images were available, then all images were selected. Parenchymal images were then converted to OME-TIFF using a recursive TiffConvert macro to engage the Bio-Formats exporter function. These converted files were then processed to binary images using imageJ functionality. Finally each binary image was analysed using the ImageJ FracLac plugin which calculates the fractal box dimension (D_{B}).

For each sheep, the mean value for each lung segment was determined. A two-way analysis of variance was conducted on the influence of the two independent variables (Lung, Treatment) on mean D_{B}. Lung was statistically significant at the 0.05 significance level. The main effect for Lung yielded an F ratio of F(3, 40)=4.15, p<0.05, indicating a significant difference between LL_Ant (M=1.70, SE=0.01), LL_Post (M=1.74, SE=0.01), RL_Ant (M=1.70, SE=0.01) and RL_Post (M=1.70, SE=0.01). The main effect for treatment yielded an F ratio of F(1, 40)=0.02, p=0.884, indicating that the effect for treatment was not significant, LMS (M=1.711, SE=0.007) and SAL (M=1.712, SE=0.007). The interaction effect was not significant, F(3, 40)=0.76, p=0.520.

It was considered these results indicated that direct exposure to radiation was associated with a significant increase in D_{B}. Treatment had no significant influence on D_{B}. As the alveolar fractal box dimension has been shown to inversely correlate with mean linear intercept in other animal models (Andersen M P, Parham A R, Waldrep J C, McKenzie W N, Dhand R. Alveolar fractal box dimension inversely correlates with mean linear intercept in mice with elastase-induced emphysema. International Journal of Chronic Obstructive Pulmonary Disease. 2012;7:235-43) it was considered the increased D_{B} seen in the radio-exposed lung of sheep treated with saline corresponded to a reduction in airspace size. 50 binary images were randomly sampled and subjected to both fractal analysis as stated above, as well as conventional assessment using STEPanizer software for stereological assessment of digital images (Tschanz S A, Burri P H, Weibel E R. A simple tool for stereological assessment of digital images: the STEPanizer. Journal of Microscopy. 2011;243(1):47-59). The data confirmed that DB was significantly negatively correlated with Lm in this randomly selected subset of images (data not shown).

The percentage of parenchyma occupied by alveolar oedema (% Area Oedema) in each Masson's trichrome-stained section was calculated. A two-way analysis of variance was conducted on the influence of two independent variables (Lung, Treatment) on Rank % Area Oedema data. Lung was statistically significant at the 0.05 significance level. The main effect for Lung yielded an F ratio of F(3, 40)=39.76, p=0.000, indicating a significant difference between LL_Ant (M=21.96, SE=1.82), LL_Post (M=41.42, SE=1.82), RL_Ant (M=18.13, SE=1.82) and RL_Post (M=16.50, SE=1.82). The main effect for treatment yielded an F ratio of F(1, 40)=0.57, p=0.456, indicating that the effect for treatment was not significant, LMS (M=25.19, SE=1.29) and SAL (M=23.81, SE=1.29). The interaction effect was not significant, F(3, 40)=0.80, p=0.503. Taken together, it was considered these results indicate that direct exposure to radiation was associated with a significant increase in % Area Oedema. These results are in agreement with those obtained using the blinded semi-quantitative scoring scheme.

Picrosirius red stain was used to enable quantification of the extent of alveolar fibrosis.

Picrosirius Red staining in lung that had not been previously exposed to radiation was evident throughout the alveolar septa (FIG. 4). In the alveolar walls the most intense staining took the form of wavy filiform fibre stretches of variable length and thickness. The septal crests and alveolar walls abutting alveolar ducts often featured more diffuse staining where individual fibres seemed teased apart into subunit fibrils. In lung that had been exposed to radiation, fibres present in the thickened alveolar septa more often appeared teased apart giving an overall subjective impression of more abundant staining. Within sections from radio-exposed lung there was often substantial variation in the extent of staining between fields, with some areas appearing identical to those from control lung sections.

Data considering the % Area of lung parenchyma occupied by collagen (red colour) in lung sections derived from the radio exposed area of the left lung (LL_Post), its contralateral control (RL_Post), and the non-radio exposed area of the left lung (LL_Ant), and its contralateral control (RL_Ant) were analysed. The highest values are found in the radio exposed lung of sheep pre-treated with saline. A two-way analysis of variance was conducted on the influence of two independent variables (Lung, Treatment) on Rank % Area Collagen (Picrosirius Red). Lung was statistically significant at the 0.05 significance level. The main effect for Lung yielded an F ratio of F(3, 40)=5.72, p=0.002, indicating a significant difference between LL_Ant (M=3.685, SE=0.750), LL_Post (M=7.787, SE=0.750), RL_Ant (M=5.562, SE=0.750) and RL_Post (M=6.751, SE=0.750). The main effect for treatment yielded an F ratio of F(1, 40)=1.51, p=0.227, indicating that the effect for treatment was not significant, LMS (M=5.509, SE=0.530) and SAL (M=6.429, SE=0.530). The interaction effect (disordinal) was significant, F(3, 40)=4.53, p=0.008, indicating that the impact of Lung depends on the Treatment. Examining the fold change in % Area of collagen found in radio exposed lung relative to its contralateral control for sheep pre-treated with saline (SAL_Rx) or LMS (LMS_Rx), and in non-radio exposed lung relative to its contralateral control for sheep pre-treated with saline (SAL_CON) or LMS (LMS_CON), determines that the greatest fold change is seen in the SAL_Rx group. One-way ANOVA of Fold change versus Group indicates that the fold change in the SAL_Rx group is significantly greater than the fold change in any other group (P=0.001). The fold changes in the other groups do not differ significantly from each other.

ASMA expression in lung that had not been previously exposed to radiation was evident at the tips of secondary septal crests abutting alveolar ducts, as well as within alveolar walls similarly adjacent to ducts (FIG. 5). The same general pattern of expression, but increased in area, was evident for radiation-exposed lung.

The average percentage area of microscopic fields in each section occupied by cells staining positively for ASMA was determined. A two-way analysis of variance was conducted on the influence of two independent variables (Lung, Treatment) on Log10 % Area ASMA. Lung was not statistically significant at the 0.05 significance level. The main effect for Lung yielded an F ratio of F(3, 40)=1.26, p=0.303, indicating no significant difference between LL_Ant (M=0.1805, SE=0.0717), LL_Post (M=0.3261, SE=0.0717), RL_Ant (M=0.3180, SE=0.0717) and RL_Post (M=0.3638, SE=0.0717). The main effect for treatment yielded an F ratio of F(1, 40)=28.45, p=0.000, indicating that the effect for treatment was significant, LMS (M=0.1060, SE=0.0507) and SAL (M=0.4882, SE=0.0507). The interaction effect did not reach significance, F(3, 40)=2.58, p=0.067.

The relationship between the % Area occupied by collagen (Picrosirius red) and that occupied by ASMA was explored for sheep pre-treated with SAL and those pre-treated with LMS. In fitting the regression model using % Area ASMA as the response variable, % Area Sirius Red as the continuous predictor, and Treatment (LMS or SAL) as the categorical predictor, the coefficient for Treatment, indicating that the vertical distance between the two regression lines, was highly significant (p=0.000). The observation that the slope of the relationship appeared similar for the two groups was examined by including the interaction term % Area Sirius Red*Treatment in the model. This confirmed that the interaction was not significant (p=0.833).

DC-LAMP expression in lung that had not been previously exposed to radiation was evident in large well-rounded cells most commonly positioned at the intersection of neighbouring alveolar walls (FIG. 6). Their appearance and position was consistent with their presumed identity as type II pneumocytes. These cells were regularly arrayed throughout the parenchyma. In contrast, in radiation-exposed lung DC-LAMP expressing cells were usually arranged in clusters. Whilst the areas between clusters were largely devoid of the regular array of expression seen in the control lung, when cells were identified at the intersection of neighbouring alveolar walls, they appeared much larger than seen in the control lung sections. Clusters of hyperplasia comprised contiguous, usually rounded but sometimes elongated or flattened, cells lining the alveolar walls. Whilst not an absolute finding, clusters were often positioned close to respiratory bronchioles and/or alveolar ducts.

ImageJ macro routines were designed to measure the percentage area (% Area) of each section occupied by DAB stain, the number of DAB particles of a given size (150 pixels2-infinity), and the average particle size.

A two-way analysis of variance was conducted on the influence of two independent variables (Lung, Treatment) on the rank-transformed averaged median DC-LAMP NND. Lung was statistically significant at the 0.05 significance level. The main effect for Lung yielded an F ratio of F(3, 40)=47.92, p=0.000, indicating a significant difference between LL_Ant (M=17.83, SE=1.95), LL_Post (M=8.67, SE=1.95), RL_Ant (M=36.25, SE=1.95) and RL_Post (M=35.25, SE=1.95). The main effect for treatment yielded an F ratio of F(1, 40)=0.36, p=0.554, indicating that the effect for treatment was not significant, LMS (M=25.08, SE=1.38) and SAL (M=23.92, SE=1.38). The interaction effect was significant, F(3, 40)=5.66, p=0.002 indicating that the lung effect depended on what treatment the sheep had received. Taken together, radiation exposure caused a decrease in DC-LAMP NND in directly exposed lung, and pre-treatment with LMS also was associated with a decrease in DC-LAMP NND in non-radioexposed left lung (LL-Ant).

Cells expressing Ki67 were only rarely observed in lung that had not been previously exposed to radiation and were variously found in the septal walls, or alveolar airspaces. Proliferating cells were more commonly identified in lung previously exposed to radiation. Occasionally these cells appeared to co-locate with cells expressing DC-LAMP (FIG. 6). Ki67-expressing cells could also be identified in perivascular fascia, and within the interstitium.

Two-way ANOVA (Table 1) indicated a significant (P=0.000) lung effect, a non-significant treatment effect (P=0.221) and a significant interaction effect (P=0.034) indicating that the lung effect depended on what treatment the sheep had received.

TABLE 1
Summary of two-way ANOVA statistics examining the influence of two factors, Lung (with four levels:
LL_Ant, LL_Post, RL_Ant, RL_Post), Treatment (with two levels: LMS, SAL), and their interaction
(Lung*Treatment)(total degrees of freedom = 40) on % Area collagen, % Area ASMA, % Area DC-LAMP
stain, the number and size of DC-LAMP positive particles, the nearest neighbour distance (NND) between
DC-LAMP positive particles, and the number of Ki67 positive particles. The F ratios and P values are
depicted in the factor columns, and the fitted means (SE mean) for each factor level is given in the
level columns. For clarity, the latter are only shown where the relevant factor effect is significant.

| | | | | | | | | | | Interaction |
|---|---|---|---|---|---|---|---|---|---|---|
| | | Factor | Levels | | | | Factor | Levels | | Lung* |
| Variable | | Lung | LL_Ant | LL_Post | RL_Ant | RL_Post | Treatment | LMS | SAL | Treatment |
| % Area | F | 5.72 | 3.685 | 7.787 | 5.562 | 6.751 | 1.51 | | | 4.53 |
| collagen | ratio | | (0.750) | (0.750) | (0.750) | (0.750) | | | | |
| | P | 0.002 | | | | | 0.227 | | | 0.008 |
| % Area | F | 1.26 | | | | | 28.45 | 0.1060 | 0.4882 | 2.58 |
| ASMA | ratio | | | | | | | (0.0507) | (0.0507) | |
| | P | 0.303 | | | | | 0.000 | | | 0.067 |
| % Area | F | 0.31 | | | | | 3.33 | | | 0.39 |
| DC- | ratio | | | | | | | | | |
| LAMP | P | 0.820 | | | | | 0.076 | | | 0.759 |
| stain | | | | | | | | | | |
| Number | F | 0.17 | | | | | 4.19 | 61.73 | 51.43 | 1.35 |
| of | ratio | | | | | | | (3.56) | (3.56) | |
| DC- | P | 0.918 | | | | | 0.047 | | | 0.273 |
| Lamp | | | | | | | | | | |
| positive | | | | | | | | | | |
| particles | | | | | | | | | | |
| Size of | F | 10.76 | 2.8953 | 2.9649 | 2.8327 | 2.8599 | 0.00 | | | 1.75 |
| DC- | ratio | | (0.0174) | (0.0174) | (0.0174) | (0.0174) | | | | |
| LAMP | P | 0.000 | | | | | 0.966 | | | 0.170 |
| positive | | | | | | | | | | |
| particles | | | | | | | | | | |
| DC- | F | 47.92 | 17.83 | 8.67 | 36.25 | 35.25 | 0.36 | | | 5.66 |
| LAMP | ratio | | (1.95) | (1.95) | (1.95) | (1.95) | | | | |
| NND | P | 0.000 | | | | | 0.554 | | | 0.002 |
| Number | F | 7.75 | 1.1208 | 1.3704 | 0.9011 | 0.9589 | 1.55 | | | 3.19 |
| of | ratio | | (0.0755) | (0.0755) | (0.0755) | (0.0755) | | | | |
| Ki67 | P | 0.000 | | | | | 0.221 | | | 0.034 |
| positive | | | | | | | | | | |
| particles | | | | | | | | | | |

The results of two-way analyses of variance conducted on the influence of the two independent variables (Lung, Treatment) on the percentage area (% Area) of DC-LAMP positive staining, the number of DC-LAMP positive particles of a given size (150 pixels²-infinity) is shown in Table 1. Two-way ANOVA (Table 1) indicated a significant lung effect (P=0.000), a non-significant treatment effect (P=0.554) and a significant interaction between these terms (P=0.002) indicating that the lung effect depended on what treatment the sheep had received. Radiation exposure caused a decrease in DC-LAMP NND in directly exposed lung, and pre-treatment with LMS also was associated with a decrease in DC-LAMP NND in non-radioexposed left lung (LL-Ant).

A two-way ANOVA (Table 1) indicated that there was a significant difference in the mean percentage collagen between the different lung segments (P=0.002). Whilst the effect for treatment was not significant (P=0.227) the interaction effect was significant (P=0.008), indicating that the impact of Lung depends on the Treatment. Examining the fold change in % Area of collagen found in radio-exposed lung relative to its contralateral control for sheep pre-treated with saline (SAL_Rx) or LMS (LMS_Rx), and in non-radio-exposed lung relative to its contralateral control for sheep pre-treated with saline (SAL_CON) or LMS (LMS_CON), determines that the fold change seen in the SAL_Rx group (FIG. 4b) significantly exceeds that seen in any other group (P=0.001).

ASMA expression: Two-way ANOVA (Table 1) indicated no significant lung effect (P=0.303), a highly significant treatment effect (P=0.000), and a non-significant interaction effect (P=0.067). The relationship between the % Area occupied by collagen (Picrosirius red) and that occupied by ASMA was explored and a significant association could be demonstrated for both groups (P=0.029 for SAL, and P=0.009 for LMS).

A two-way analysis of variance was conducted on the influence of two independent variables (Lung, Treatment) on Log10% Ki67 count. Lung was statistically significant at the 0.05 significance level. The main effect for Lung yielded an F ratio of F(3, 40)=7.75, p=0.000, indicating a significant difference between LL_Ant (M=1.1208, SE=0.0755), LL_Post (M=1.3704, SE=0.0755), RL_Ant (M=0.9011, SE=0.0755) and RL_Post (M=0.9589, SE=0.0755). The main effect for treatment yielded an F ratio of F(1, 40)=1.55, p=0.221, indicating that the effect for treatment was not significant, LMS (M=1.1347, SE=0.0534) and SAL (M=1.0409, SE=0.0534). The interaction effect was significant, F(3, 40)=3.19, p=0.034 indicating that the lung effect depended on what treatment the sheep had received.

A model system in sheep was used in this study. It is proposed this model demonstrates similar findings as would be expected to be observed in humans. The consistent histopathological features associated with lung irradiation in this study, which developed within 37 days of the first exposure to radiation (within 23 days of the last exposure), were intraalveolar oedema, alveolar fibrosis, interstitial pneumonia, and pneumocyte type II hyperplasia. Whilst observations of the earliest effects of radiation to human lungs are unavailable Gross (Gross N J. Pulmonary effects of radiation therapy. Ann Intern Med. 1977; 86(1):81-92) in reviewing autopsy studies of humans dying of pneumonitis 4-12 weeks after radiotherapy also found alveolar septa thickened with oedema, cell infiltrates and the laying down of connective tissue, together with atypia, hyperplasia and desquamation of alveolar epithelial cells and the presence of hyaline membranes The early appearance of alveolar septal fibrosis is not an isolated finding. Indeed, Jennings & Arden (Jennings FL, Arden A. Development of radiation pneumonitis. Time and dose factors. Arch Pathol. 1962; 74:351-60) found that alveolar septal fibrosis could be seen in some instances less than 30 days after radiation exposure. Bennett et al (Bennett DE, Million RR, Ackerman LV. Bilateral radiation pneumonitis, a complication of the radiotherapy of bronchogenic carcinoma. (Report and analysis of seven cases with autopsy). Cancer. 1969; 23(5):1001-18), in analysing seven autopsies in which bilateral radiation pneumonitis following radiotherapy was the primary or major contributory cause of death, found alveolar septal fibrosis to be a prominent feature in five of the cases, with these patients dying between 40 and 95 days after completion of radiotherapy.

Despite sharing aspects of pathology with patients dying of radiation pneumonitis the sheep in the present study demonstrated no clinically overt adverse effect as a consequence of radiation exposure. It is considered this is a function of the fractionated dose regime and volume targeted in the present study. Indeed it has previously been shown that sheep will indeed develop radiation pneumonitis typical of that seen in humans given sufficient dose and lung volume targeted (Ohkuda K, Abe Y, Ohnuki T, Koike K, Watanabe S, Nitta S, et al. Effects of irradiation on the pulmonary vascular fluid and protein exchange. The Tohoku journal of experimental medicine. 1982; 138(3):309-12; Perkett E A, Brigham K L, Meyrick B. Increased vasoreactivity and chronic pulmonary hypertension following thoracic irradiation in sheep. J Appl Physiol. 1986; 61(5):1875-81; Loyd J E, Bolds J M, Sheller J R, Duke S S, Gillette A W, Malcolm A W, et al. Acute effects of thoracic irradiation on lung function and structure in awake sheep. J Appl Physiol. 1987; 62(1):208-18; Guerry-Force M L, Perkett E A, Brigham K L, Meyrick B. Early structural changes in sheep lung following thoracic irradiation. Radiat Res. 1988; 114(1):138-53; and Tillman B F, Loyd J E, Malcolm A W, Holm B A, Brigham K L. Unilateral radiation pneumonitis in sheep: physiological changes and bronchoalveolar lavage. J Appl Physiol. 1989; 66(3):1273-9).

Unilateral single fraction irradiation (30 Gy) of the sheep thorax produced radiation pneumonitis typical of the syndrome in humans at 4 weeks after irradiation, and whole lung irradiation (15 Gy) of sheep resulted in the development of dyspnoea three weeks after exposure that continued to progress until the animals were killed at 4 weeks. The inventors consider that sheep replicate many aspects of the early human response to lung irradiation, and that substantial pathology including alveolar fibrosis develops in sheep subjected to a radiotherapy regime (30 Gy/5 F/2 wk), which bears resemblance to palliative regimens routinely applied to patients with metastatic lung cancer, and some patients with locally advanced disease.

Whilst peribronchial and peribronchiolar inflammatory cells, comprising mostly plasma cells and lymphocytes, were relatively frequently identified in irradiated lung, the inventors found no evidence of a radiation influence on bronchial epithelial cytokine expression. Previous clinical studies which have assessed changes in plasma cytokine concentration during radiotherapy for lung cancer have demonstrated increased circulating TGF-β1, IL-6 and IL-10, and MCP-3, δMIP-1a, and IP-10. The specific cellular source of these cytokines has not been definitively ascertained.

Radiation was not considered to influence bronchial epithelial cytokine expression in this model, and is therefore unlikely to prove a useful indicator of the effect of radiation exposure in this context.

A significant increase in the percentage of parenchyma affected by oedema in radiation-exposed lung was determined by the inventors. In the method used by the inventors—manually annotating low power whole slide images of Masson Trichrome-stained sections—it is only sufficiently sensitive to identify lung areas where there is complete alveolar flooding with oedema fluid that takes up the stain. Hence the method likely underestimates the true extent of oedema and may be susceptible to variable stain uptake as a consequence of variation in the composition of the oedema fluid.

In radiation-exposed lung of sheep treated with saline, the inventors demonstrated a median fold change in the percentage area of parenchyma occupied by Sirius red-stained collagen (relative to the contralateral control lung) of 1.68 (range: 1.25-4.01).

The fibroblast populations in the alveolar mesenchyme are responsible for producing tropocollagen, the molecular component of collagen fibres, and the ground substance that fills the spaces between the cells and various fibres in the interstitial space. A particular population of differentiated fibroblasts comprise the myofibroblasts which are characterised by their expression of ASMA, as well as their ability to contract in a smooth muscle cell-like manner. Myofibroblasts play a fundamental role in alveologenesis.

In healthy lung sections ASMA expression is recognised at the tips of secondary septal crests, representing the cross-sectioned ridges running between the alveoli surrounding the alveolar ducts.

The structure of the alveolar interstitial matrix is significantly compromised as a consequence of radiation exposure. Central among the growth factors that co-ordinate matrix tissue re-modelling is transforming growth factor-beta. This growth factor, which is ubiquitously expressed by all cells and tissues within the body, promotes extracellular matrix (ECM) deposition by stimulating different collagen, elastin, fibronectin and proteoglycan genes to produce ECM components. During synthesis two TGF-β precursor proteins form a dimer which is then cleaved by furin into two products, the first being a latency-associated peptide (LAP) and the other being mature TGF-β. These products thereafter maintain a non-covalent association forming a complex referred to as the small latent complex, which in turn covalently links to latent TGF-β binding protein (LTBP) to form the large latent complex which is then secreted and incorporated into the extracellular matrix as an inactive molecule. In addition to physical influences such as acidification or temperature changes TGF-β can be activated by proteases, by reactive oxygen species, or by interacting with thrombospondin or the αv-containing integrins (αvβ5, αvβ6, and αvβ8). Integrin αvβ5 is expressed by airway epithelial cells, endothelial cells, fibroblasts and monocytes in the lung, integrin αvβ8 by airway epithelial basal cells, and αvβ6 by airway epithelial cells. Activated TGF-β can then interact with its receptors leading to phosphorylation of transcription factors Smad2 and/or Smad3 which in turn associate and form a complex with Smad4 before translocating to the nucleus to influence the transcription of target genes and the production of ECM components. In a rat model of radiation-induced lung injury protein expression of integrin αvβ6, TGF-β1, TβRII, Smad3, and p-Smad2/3 was undetectable in the normal alveolar epithelium but increased in association with lung fibrosis six months after radiation exposure.

The present result in significant positive association between ASMA expression and collagen deposition in lung sections from sheep exposed to radiation.

Analysis by the inventors of expression of DC-LAMP found that radiation exposure was associated with clustering and an increase in size of DC-LAMP-positive cells. Type II pneumocytes are considered to proliferate in response to injury and serve as progenitors for replacing lost or damaged type I pneumocytes lining the alveolar surface.

Type II cells are well-recognised to be early susceptible targets of radiation effects and the inventors found that their reduced presence in their normal niches at alveolar corners was associated with an increase in size of the remaining cells found in these sites.

Pre-treating sheep with nebulised lamellar body compositions of the invention prior to each radiation exposure abrogated the increase in collagen seen in the PTV of sheep pre-treated with saline.

Lamellar body composition pre-treatment significantly increased the number of DC-LAMP positive cells in the lung relative to sheep pre-treated with saline, which contributed to a non-significant trend (P=0.067) towards an increase in the DC-LAMP area percentage. The lamellar body composition, in influencing the ability of type II cells to manage the interstitium, is proposed to change the proportion of myofibroblasts in this compartment in health, which would explain the significant treatment effect on ASMA.

Pre-treatment with the lamellar body composition discussed herein was associated with clustering of DC-LAMP positive cells and an increase in Ki67 count in non-radioexposed left lung (giving rise to significant interaction effects). The inventors considered the proximity of the anterior blocks to the cranial margin of the PTV might be a factor in dictating this difference between the treatment groups. Anterior blocks were selected as follows: The block containing the cranial margin of the PTV was identified. Progressing cranially, its immediate neighbour was disregarded and the next block along selected as LL (or RL)_Ant. As this procedure was consistent it was assumed that any variation in the spatial relationship between the selected blocks and the cranial margin of the PTV would be randomly spread between the SAL and lamellar body groups. However, when the inventors retrospectively examined these distances, they determined that there was a significant difference between the groups in terms of the distance of the “Ant” section from the cranial margin of the PTV—the lamellar body sections were approximately 7 mm closer (data not shown). Further, when the relationship between LL_Ant and RL_Ant DC-LAMP NND and Ki67 cell counts was expressed as absolute difference (LL-RL), and fold-change (1+((LL-RL)/RL) respectively, and compared to the distance of the section from the cranial edge of the PTV, significant correlations were found which would appear to explain the observation that pre-treatment with lamellar body composition was associated with a decrease in DC-LAMP NND and an increase in Ki67 count in non-radioexposed left lung (data not shown). This suggests a ramped decline in the biological effect of radiation (at least in terms of DC-LAMP cell clustering and cell proliferation) that extends beyond the margins of the PTV in this model.

TGF-β1 Model

An established in vitro model of myofibroblast activation using primary fibroblasts derived from the lungs of healthy donors and patients with idiopathic pulmonary fibrosis (IPF) was selected to investigate the anti-fibrotic effect of prepared lamellar body compositions outlined in the table below. The model involves a 96-well assay in which cells are treated with TGF-β1 in order to stimulate the differentiation of fibroblasts to myofibroblasts allowing for high-throughput screening of anti-fibrotic compounds using cells from multiple donors.

| Formulation/ | | |
|---|---|---|
| No | Lipid Composition | Mass Ratio |
| LMS-611 | DOPC/ESM/DOPE/DOPS/HSPI/Chol | 55.5/19.4/8.2/4.1/3.1/10.1 |
| | (LMS-611) | |
| 1 | DOPC/ESM/DOPE/DOPS/HSPI/Chol | 55.1/19.4/8.2/4.1/3.1/10.1 |
| | (LMS-611 downsized to ca 125 nm) | |
| 2 | DOPC/ESM/DOPE/DOPG/Chol | 55.3/19.4/8.2/6.8/10.1 |
| 3 | DOPC/ESM/DOPE/Chol | 62.1/19.5/8.2/10.1 |
| 4 | DOPC/ESM/DOPS/Chol | 61.7/19.4/8.9/10.0 |
| 8 | DOPC/ESM/DOPE/DOPS/HSPI/Chol/LysoPC | 54.5/19.2/8.1/4.0/3.1/10.0/1.1 |

For the purposes of this study, fibroblasts from healthy donors were used at Passage 4. On Day 0, cells were seeded in 96-well plates and incubated at 37° C. for 48 hours. On Day 2, the cell culture medium was refreshed. On Day 5, the cells were treated with an 8-point concentration curve of each lamellar body formulation. The concentration curve was generated by two-fold serial dilutions of a top total lipid concentration of 1.5 mg/ml diluted with 0.9% saline solution. One hour post-treatment, the cells were stimulated with 1.25 ng/ml TGF-β1. The cells were incubated for a further 72 hours.

To confirm the assay was functional, 1nM SB-525334 was used a positive control for anti-fibrotic activity. SB525334 is an inhibitor of ALK5 (TGF-β Receptor 2) and inhibits TGF-β1 signalling. Cells were treated with SB-525334 for one hour in parallel to the lamellar body treatments. Cells treated with 0.1% DMSO or 0.9% saline (3% of the final volume per well) served as vehicle controls for the SB-525334 and lamellar body formulations, respectively. Nintedanib, a drug clinically approved for treatment of IPF, was also used as a reference compound against which to compare the efficacy of lamellar body formulations. An 8-point concentration curve was used, with a top concentration of 10 μM.

On Day 8, 72 hours post-stimulation with TGF-β1, cells were fixed with 4% formaldehyde.

Analysis of myofibroblast differentiation in response to TGF-β1 was carried out by means of confocal microscopy imaging of fluorescent staining of alpha smooth muscle actin (αSMA) and DAPI staining of the cell nuclei. This method measures the density times the area (D×A) of αSMA staining. The number of nuclei staining positive for DAPI serves as an indicator of possible compound cytotoxicity.

The assay was deemed to be suitable for the analysis of potential anti-fibrotic effects of LMS-611 composition and other novel formulations of lamellar bodies. Determining formulations (1, 2 and 8) induced a partial inhibition of TGF-β1-induced αSMA, the DOPS-enriched Formulation 4 caused a full dose-dependent inhibition in upregulation of αSMA in response to TGF-β1 stimulation.

The following table summarises the formulations tested and whether or not they were able to demonstrate an antifibrotic effect in this FMT assay model.

| Formulation | | | Antifibrotic |
|---|---|---|---|
| No | Lipid Composition | Mass Ratio | Effect * |
| LMS-611 | DOPC/ESM/DOPE/DOPS/HSPI/Chol (LMS-611) | 55.5/19.4/8.2/4.1/3.1/10.1 | Inconclusive** |
| 1 | DOPC/ESM/DOPE/DOPS/HSPI/Chol (LMS-611 | 55.1/19.4/8.2/4.1/3.1/10.1 | Y |
| | downsized to ca 125 nm) | | |
| 2 | DOPC/ESM/DOPE/DOPG/Chol | 55.3/19.4/8.2/6.8/10.1 | Y |
| 3 | DOPC/ESM/DOPE/Chol | 62.1/19.5/8.2/10.1 | N |
| 4 | DOPC/ESM/DOPS/Chol | 61.7/19.4/8.9/10.0 | Y |
| 8 | DOPC/ESM/DOPE/DOPS/HSPI/Chol/LysoPC | 54.5/19.2/8.1/4.0/3.1/10.0/1.1 | Y |

* based on results from 2 human donors
**results from one donor showed inhibition however data from a second donor did not.

Cell Entry Model

To elucidate cellular interaction, all prepared formulations, of lamellar body formulations, were labelled with 1,1′-dioctadecyl-3,3,3′,3′-tetramethylindocarbocyanine perchlorate (Dil), a lipophilic non exchangeable fluorescent lipid label. Cellular interactions were measured by flow cytometry using trypan blue, a quencher of extracellular fluorescence, to discriminate between cell association and internalisation of the vesicles. This is a standard technique as described earlier (Sahlin et al., 1983; Feldmann et al., 2017). All variants were tested in HeLa cells with some also tested in A549 cells. The selected cell lines were taken as model cell lines to illustrate that the formulations are taken up by cells.

Preparation Methods

Dual centrifugation was used to effectively homogenise a lipid/water blend to form a vesicular phospholipid gel (VPG) and after subsequent dilution of the VPG to prepare lamellar body formulations. This process method is described in (Massing, U., Ingebrigtsen, S. G., S̆kalko-Basnet, N., Holster, A. M., 2017. Dual Centrifugation—A Novel “in-vial” Liposome Processing Technique, in: Catala, A. (Ed.), Liposomes. InTech. https://doi.org/10.5772/intechopen.68523). Other methods such as extrusion and microfluidisation can be used to prepare lamellar body aqueous dispersions as well.

Lipid mixtures were prepared by mixing of dissolved lipids with 0.5 mol % Dil with respect to total lipid amount in a suitable organic solvent or solvent mixture followed by removal of the solvent by drying under vacuum. Aqueous dispersions of the lamellar body formulations were prepared by hydration of the dry lipid film in 250 mM sucrose and 25 mM sodium chloride and processed in a dual centrifuge (ZentriMix 380R, Andreas Hettich GmbH, Germany) at 1200 rpm, 15° C. for 20 min. The resulting vesicular phospholipid gel (VPG) was diluted with aqueous medium and processed again at 1200 rpm, 15° C. for 5 min. Ceramic beads were used as mixing aids in the vials. Finally, the formulations were further diluted to the required concentration.

Characterisation of Preparations

Phospholipid concentrations after extrusion were determined using the Bartlett assay (Bartlett, 1959 Bartlett, G. R., 1959. Phosphorus assay in column chromatography. J. Biol. Chem. 234, 466-468.). For all formulations, vesicle size and size range were measured. The composition, size range and zeta potential of the tested lamellar body formulations are provided in the following Table (Characteristics of Different Formulations—Note: All formulations were labelled with 0.7 weight % of Dil.).

Characteristics of Different Formulations

| | | | | Zeta |
|---|---|---|---|---|
| | | Identifica- | Size | Potential |
| Lipids | Ratio w/w | tion Label | (nm) | (mV) |
| DOPC/ESM/DOPE/DOPS/ | 54.8/19.3/8.1/4/3.1/10 | LMS-611 | 123 | −24 |
| HSPI/Chol | | | | |
| DOPC/Chol | 89.5/9.8 | Not Applicable, | 124 | 0 |
| | | used as | | |
| | | Neutral | | |
| | | Control | | |
| DOPC/ESM/DOPE/DOPG// | 54.9/19.4/8.2/6.8/10 | LMS_DOPG | 125 | −21 |
| Chol | | | | |
| DOPC/ESM/DOPE/DOPS/ | 54.9/19.4/8.2/4/2.9/10 | LMS_DOPS/DSPG | 126 | −24 |
| DSPG//Chol | | | | |
| DOPC/ESM/DOPE/DSPG// | 54.9/19.4/8.2/6.9/10 | LMS_DSPG | 127 | −24 |
| Chol | | | | |
| DOPC/ESM/DOPS/Chol | 69.5/14.9/9.9/5 | 009 | 113 | −25 |
| DPPC/DOPE/DOPS/Chol | 26.5/32.3/29.3/11.2 | 010 | 131 | −58 |
| DOPC/DOPE/DOPS/Chol | 27.9/31.7/28.8/11 | 011 | 108 | −55 |
| DOPC/DOPS/Chol | 60.3/28.2/10.8 | 012 | 110 | −53 |

In vitro Uptake Experiments

For cellular uptake experiments 6.5·10⁴ cells (A549 and HeLa) were seeded into the wells of a 24 well plate. After 24 hours the medium was changed and cells were incubated with the lamellar body formulations (0.15 mM) for 2 hours at 37° C. Cells were then analysed using a flow cytometer (BD LSRFortessa™ with BD FACSDiva software 8.01, Becton Dickinson, Germany) to assess the Dil fluorescence (excitation 561 nm, emission 585/15 nm).

A 0.08% aqueous solution of trypan blue, a quencher of the fluorescence of Dil, which cannot permeate cells, was used to discriminate between association and uptake of the lamellar bodies as described previously (Sahlin, S., Hed, J., Rundquist, I., 1983. Differentiation between attached and ingested immune complexes by a fluorescence quenching cytofluorometric assay. J. Immunol. Methods 60, 115-124.; Feldmann, D. P., Xie, Y., Jones, S. K., Yu, D., Moszczynska, A., Merkel, O. M., 2017. The impact of microfluidic mixing of triblock micelleplexes on in vitro/in vivo gene silencing and intracellular trafficking. Nanotechnology 28, 224001. https://doi.org/10.1088/1361-6528/aa6d15). The correction for spectral overlap of Dil and trypan blue was carried out with BD FACSDiva software.

Uptake experiments were analysed by normalising the Dil fluorescence intensities of the tested formulations to the Dil fluorescence of neutral control liposomes (DOPC/Chol).

Results

Lamellar body vesicle formulation LMS-611 was able to enter both cell lines. Fluorescence intensities were 40-fold (A549 and HeLa) higher compared to DOPC/Chol control liposomes. Additionally, in both cell lines LMS-611 lamellar body formulations were internalised to a high degree (93% of fluorescence was not quenched by trypan blue). The lamellar body formulations all demonstrated significantly greater cell entry than the DOPC/Chol control liposomes.

FIG. 9 depicts cellular association (red bars) and internalisation (blue bars) of formulations in A549 cells (A) and HeLa cells (B) after a 2 hour incubation period. The fold change of Dil fluorescence is normalised to neutral DOPC/Chol liposomes.

Cellular entry of lamellar body formulations was observed with formulations prepared from six, five or four or three lipids, containing negatively charged lipids. The phospholipids in the formulations comprise esterified saturated and unsaturated fatty acids. The example illustrates that lamellar body formulations are suitable to be taken up in general by cells. Thus, it is considered these compositions can act at the site of pathology within the cell to minimise or prophylactically treat fibrotic conditions.

Although the invention has been particularly shown and described with reference to particular examples, it will be understood by those skilled in the art that various changes in the form and details may be made therein without departing from the scope of the present invention.

1. A lamellar body composition for use in the prophylactic treatment of fibrotic or pro-fibrotic conditions, optionally wherein the conditions are selected from conditions of the lung, skin, gastrointestinal system, genitourinary system, heart, peritoneum, kidney, liver, and mucosa.

2. A lamellar body composition for use in the prophylactic treatment of fibrotic or pro-fibrotic conditions as claimed in claim 1 formulated for administration via the airway to the epithelium of the lower airways for the prevention and/or treatment of distal lung injury due to direct or indirect injury optionally wherein the lung injury is selected from sepsis, ventilator-induced lung injury, ischemia/reperfusion, hyperoxia, ALI, ARDS, or conditions caused by irradiation of the lower neck, thoracic structures or chest wall.

3. The lamellar body composition for use in the treatment of fibrotic or pro-fibrotic conditions formulated for administration via the airway to the epithelium of the lower airways for at least one of the prevention and treatment of distal lung injury due to direct or indirect injury as claimed in claim 2 wherein the lung injury is selected from conditions caused by irradiation of the lower neck, thoracic structures or chest wall.

4. The lamellar body composition for use in the prophylactic treatment of fibrotic or pro-fibrotic conditions of any one of the preceding claims wherein the lamellar bodies in the lamellar body composition are sized at less than 250 nm.

5. The lamellar body composition for use in the treatment of fibrotic or pro-fibrotic conditions of any one of the preceding claims wherein the lamellar bodies are provided as droplets with an average size about 1.5 microns.

6. The lamellar body composition for use in the prophylactic treatment of fibrotic or pro-fibrotic conditions of any one of the preceding claims comprising phosphatidylcholine, cholesterol and optionally at least one phospholipid selected from phosphatidyl serine, phosphatidyl glycerol and phosphatidyl inositol to provide an anionic lamellar body.

7. The lamellar body composition for use in the prophylactic treatment of fibrotic or pro-fibrotic conditions of any of the preceding claims comprising phosphatidylcholine, cholesterol and phosphatidyl serine.

8. The lamellar body composition for use in the prophylactic treatment of fibrotic or pro-fibrotic conditions of any of the preceding claims comprising about 44-70% phosphatidylcholine, about 2-18% phosphatidyl serine, and about 4-12% cholesterol by weight and optionally a further lipid.

9. The lamellar body composition for use in the prophylactic treatment of fibrotic or pro-fibrotic conditions of any of the preceding claims comprising about 44-70% phosphatidylcholine, about 15-23% sphingomyelin, about 6-10% phosphatidyl ethanolamine, about 2-15% phosphatidyl serine, about 2-4% phosphatidyl inositol and about 4-12% cholesterol by weight.

10. The lamellar body composition for use in the prophylactic treatment of fibrotic or pro-fibrotic conditions of any of the preceding claims comprising about 54% phosphatidylcholine, about 19% sphingomyelin, about 8% phosphatidyl ethanolamine, about 4% phosphatidyl serine, about 3% phosphatidyl inositol and about 10% cholesterol by weight, optionally comprising about 2% by weight lysophosphatidyl choline.

11. A method of supplementing lung surfactant in a mammalian subject with depleted lung surfactant, comprising administering to the subject a lamellar body composition comprising phosphatidylcholine, cholesterol and optionally at least one phospholipid selected from phosphatidyl serine, phosphatidyl glycerol and phosphatidyl inositol to provide an anionic lamellar body formulated for administration via the airway to the epithelium of the lower airways in a treatment effective amount.

12. A method of treating lung injury in a mammalian subject in need thereof, comprising administering to said subject a lamellar body composition comprising phosphatidylcholine, cholesterol and optionally at least one phospholipid selected from phosphatidyl serine, phosphatidyl glycerol and phosphatidyl inositol to provide an anionic lamellar body formulated for administration via the airway to the epithelium of the lower airways, optionally wherein the lung injury is selected from sepsis, ventilator-induced lung injury, ischemia/reperfusion, hyperoxia, ALI, ARDS, or radiation treatment

13. The method of treating a lung injury as claimed in claim 11 or 12, wherein said lung injury is a radiation treatment injury caused by alpha radiation, beta radiation, neutron radiation, or gamma radiation, optionally wherein said radiation injury is radiation pneumonitis or radiation-induced lung injury, or a fibrotic pulmonary condition caused by radiation.

14. The method of any one of claims 11 to 13, wherein said administering step is carried out as a single dose of said treatment effective amount, optionally wherein said administering step is carried out by inhalation administration.

15. The method of any one of claims 11 to 14, wherein said administering step is carried out within 1, 2 or 3 days of said ionizing radiation injury.

16. The method of any of claims 11 to 14 wherein said administering step is carried out 1, 2 or 3 days prior to treatment of the subject with ionizing radiation.
